(19) United States
(12) Patent Application Publication
(10) Pub. No.: US 2003/0087942 A1
(43) Pub. Date: May 8, 2003

(54) Method for the treatment and prevention of cachexia

(75) Inventor: Louis Muglia, St. Louis, MO (US)

Correspondence Address: SONNENSCHEIN NATH & ROSENTHAL, P.O. BOX 061080, WACKER DRIVE STATION, CHICAGO, IL, 60606-1080, US

(73) Assignee: Washington University

(21) Appl. No.: 10/231,553

(22) Filed: Aug. 30, 2002

Related U.S. Application Data
(60) Provisional application No. 60/316,004, filed on Aug. 31, 2001.

(51) Int. Cl. A61K 31/421; A61K 31/415

(52) U.S. Cl. 514/374; 514/406

(57) ABSTRACT

Cachexia, including anorexia and other forms of weight loss, is a frequent complication of acute and chronic infections, and result from induction of cytokines, prostaglandins, and other inflammatory mediators that are critical for pathogen elimination. The present invention includes methods for the treatment or prevention of cachexic conditions while maintaining the production of factors essential for infection control through the administration of an effective amount of a cyclooxygenase-2 selective inhibiting compound.

### PRIORITY This application claims the benefit of U.S. provisional application Serial No. 60/316,004 filed Aug. 31, 2001. That application is incorporated by reference as if restated here in full.

### BACKGROUND

Activation of the immune system during acute bacterial or viral infections results in generation of cytokines and other inflammatory mediators that are essential for infection control. These inflammatory mediators result in mononuclear cell chemotaxis and activation, and local changes in blood flow and vascular permeability that facilitate pathogen eradication. In addition to these beneficial actions, factors induced during infection or other systemic inflammation also exhibit detrimental actions. One important consequence of inflammation is anorexia, accompanied by acute weight loss. This phenomenon is described, for example, by Langhans and Hrupka (Neuropeptides 33: 415-24, 1999). Additional work in this area was performed by Langhans and Hrupka (Neuropeptides 33: 415-24, 1999). This phenomenon is further described by Sonti et al. (Am J Physiol Regulatory Integrative Comp Physiol 270: R1394-402, 1996). With severe or prolonged infection or inflammation, these changes in nutrition can adversely affect resolution of infection, wound healing, and growth.

Lipopolysaccharide (LPS), a major component of the outer cell wall of gram-negative bacteria, has been extensively utilized as a model for acute sepsis. Similar to bacterial infection, LPS administration results in fever, robust cytokine production, and anorexia in rodents. See for example, Li et al., Brain Res 825: 86-94, 1999. As a further example, see Porter et al., Am J Physiol Regulatory Integrative Comp Physiol 279: R2113-20, 2000. Inhibition of cytokine production or action after LPS, specifically TNF-α and IL-1β, attenuates LPS-induced anorexia. See for example Porter et al., Am J Physiol Regulatory Integrative Comp Physiol 274: R741-5, 1998. However, inhibition of these proximal mediators of the inflammatory cascade would be expected to compromise survival in the setting of a live, replicating pathogen as suggested by the increased susceptibility of mice with genetic deficiency of TNF receptor 1, IL-6, and interferon-γ to infection with bacterial agents such as Listeria monocytogenes. See for example Rothe et al., Nature 364: 798-802, 1993. Further work in this area was performed by Kopf et al., Nature 369: 339-42, 1994.

Inhibition of prostaglandin production has also been shown to attenuate the anorectic response to LPS or IL-1β, but the mechanism by which this occurs and the consequences for generation of a protective inflammatory response remain unclear. For example, Langhans et al. (Physiol Behav 46: 535-9, 1989) described experiments in which the non-selective cyclooxygenase inhibitor indomethacin attenuated anorectic response caused by LPS. Shimomura et al. (Life Sci 51: 1419-26, 1992) described the use of a cyclooxygenase and lipoxygenase inhibitor to partially attenuate anorexia induced by IL-1β. The first committed step in prostaglandin synthesis, catalyzed by prostaglandin H synthases, or cyclooxygenases, has served as an important therapeutic target for treatment of inflammatory disease (Smith and Dewitt, Advances in Immunology 62: 167-215, 1996). Recently developed non-steroidal anti-inflammatory drugs (NSAIDs) are capable of selectively inhibiting the function of one of the two cyclooxygenase (COX) isoforms COX-1 or COX-2. For example, Masferrer et al. describe the selective inhibition of COX-2 in vivo is anti-inflammatory and nonulcerogenic (Proc Natl Acad Sci USA 91: 3228-3232, 1994). Masferrer et al. further describe COX-2 inhibitors and their properties in Gastroenterology Clinics of North America 25: 363-72, 1996.

Traditional NSAIDs may actually increase, rather than decrease proximal events in the response during inflammation, such as induction of TNF-α Molloy et al., J. Immunal, 151: 2142-2149, 1993.

The phosphodiesterase inhibitor pentoxyphylline has been shown to attenuate inflammation-induced anorexia (Langhans, Nutrition 12: 303-15, 1996). There is at least one report of a calcium channel blocker attenuating inflammation-induced anorexia (Langhans et al., Physiol Behav 46: 535-9, 1989).

Breder and Saper (Brain Res 713: 64-9, 1996) reported that following LPS administration, COX-2 mRNA and protein are induced within the CNS, predominantly in vascular endothelium, though also in specific neuronal populations. Additional work in this area was performed by Matsumura et al., J Neurosci 18: 6279-87, 1998. COX-2 has also been demonstrated to be the COX isoform essential for generation of the febrile response after systemic inflammation (Matsumura et al., Brain Res 825: 86-94, 1999).

### SUMMARY OF THE INVENTION

It will be advantageous to provide methods of treating or preventing cachexic conditions in patients. In this regard, the ability to selectively inhibit the anorectic response while preserving the actions of inflammatory mediators necessary for pathogen elimination would be a very useful addition to antimicrobial therapy of infection. The present invention provides a method for attenuating the anorectic response accompanying immune system activation without altering cytokine or adrenal responses. Accordingly, the present invention provides a method for the treatment or prevention of a cachexic condition in a subject in need of such treatment or prevention, wherein the method comprises administering to the subject a cachexic condition treating- or preventing-effective amount of a cyclooxygenase-2 selective inhibiting compound, thereby treating or preventing the cachexic condition.

### DESCRIPTION OF THE FIGURES

FIG. 1 is a graph showing changes in subject body weight over time for vehicle versus LPS treatment illustrating that pharmacological prostaglandin synthesis inhibition attenuates LPS-induced weight loss.

FIG. 1A shows body weight changes after administration of vehicle (n=7), indomethacin (Indo, n=4), or isoform-specific COX inhibitors (n=4-5) without LPS administration.

FIG. 1B shows body weight changes after administration of vehicle (n=11), Indo (n=5), or isoform-specific COX inhibitors (n=10-12) with LPS administration. Statistically significant changes: day 1, P<0.001, vehicle/LPS vs. Compound 1/LPS; P<0.05, Compound 1/LPS vs. Compound 2/LPS; day 2, P<0.05 Compound 1/LPS vs. Compound 2/LPS; day 3, P=0.01, vehicle or Compound 1/LPS vs. Compound 2/LPS.

FIG. 2 is a graph showing changes in body weight over time for COX-2 treated mice illustrating that genetic prostaglandin synthesis inhibition attenuates LPS-induced weight loss. COX-2 KO mice tended to have less weight loss than WT mice one day after LPS administration (P=0.08), and significantly less weight loss than COX-1 KO mice both one day (P<0.01) and two days (P<0.01) after LPS administration (n=5-7 per group).

FIG. 3 is bar graphs showing appetite and metabolic rate changes after LPS administration with COX-2 inhibition.

FIG. 3A shows food intake over the 24 h period following LPS administration. *P<0.01 vs. vehicle/LPS and vehicle/vehicle (n=9-10 per group).

FIG. 3B shows body weight changes with overnight food withdrawal and LPS administration (n=5 per group). *P<0.005 vs. vehicle/vehicle; **P<0.05 vs. vehicle/vehicle.

FIG. 4 is bar graphs showing plasma hormone induction after LPS administration.

FIG. 4A shows plasma IL-6 measurements after LPS administration (n=3-5 per group). Time 0 and vehicle/vehicle IL-6 concentrations were below the assay detection limit of 0.1 ng/ml.

FIG. 4B shows plasma corticosterone measurements after LPS administration (n=4-5 per group). *P<0.001 vs. vehicle/LPS and Compound 1/LPS at same time point.

FIG. 4C shows plasma leptin concentration after LPS administration (n=5 per group). *P<0.05 vs. vehicle/LPS and Compound 1/LPS at 3 h; **P<0.01 vs. vehicle/LPS and Compound 1/LPS at 16 h.

FIG. 5 shows in situ hybridization analysis of hypothalamic CRH and NPY.

FIG. 5A shows representative autoradiograms of coronal sections through the hypothalamic paraventricular nucleus (pvn) for CRH and arcuate nucleus (arc) for NPY mRNA detection. The central nucleus of the amygdala (am) also demonstrates CRH mRNA hybridization. V represents vehicle.

FIG. 5B shows quantitative densitometric analysis of CRH and NPY in situ autoradiograms (n=3 per group). *P<0.05 vs. vehicle/vehicle CRH.

### DETAILED DESCRIPTION OF THE INVENTION

As used herein, the term “cachexia” or “cachexic condition” means a profound and marked state of malnutrition, frequently marked by significant weight loss. In one embodiment the present invention provides a method for the treatment or prevention of a cachexic condition in a subject in need of such treatment or prevention, wherein the method comprises administering to the subject a cachexic condition treating- or preventing-effective amount of a cyclooxygenase-2 selective inhibiting compound, thereby treating or preventing the cachexic condition. As used herein, the term “compound” comprises the specific compounds identified as well as all related pharmaceutically acceptable compositions including precursors, compound sources, pro-drugs, and salts thereof.

In one preferred embodiment, the cyclooxygenase-2 selective inhibiting compound is a tricyclic cyclooxygenase-2 selective inhibiting compound. For example, the tricyclic cyclooxygenase-2 selective inhibiting compound can be celecoxib, deracoxib, valdecoxib, parecoxib, rofecoxib, etoricoxib, or 4-(4-cyclohexyl-2-methyloxazol-5-yl)-2-fluorobenzenesulfonamide. In another preferred embodiment, the cyclooxygenase-2 selective inhibiting compound is a benzopyran cyclooxygenase-2 inhibiting compound. In still another preferred embodiment, the cyclooxygenase-2 selective inhibiting compound is 5-ethyl-2-(2′,4′-dichloro-6′-methylanilino)phenylacetic acid. In another embodiment, the cyclooxygenase-2 selective inhibiting compound can be selected from the group consisting of lumiracoxib, celecoxib; deracoxib; valdecoxib; parecoxib; rofecoxib; etoricoxib; meloxicam, 4-(4-cyclohexyl-2-methyloxazol-5-yl)-2-fluorobenzenesulfonamide; 6-[[5-(4-chlorobenzoyl)- 1,4-dimethyl- 1H-pyrrol-2-yl]methyl]-3(2H)-pyridazinone; N-(4-nitro-2-phenoxyphenyl)methanesulfonamide; 3-(3,4-difluorophenoxy)-5,5-dimethyl-4-[4-(methylsulfonyl)phenyl]-2(5H)-furanone; N-[6-[(2,4-difluorophenyl)thio]-2,3-dihydro-1-oxo-1H-inden-5-yl]methanesulfonamide; N-[2-(cyclohexyloxy)-4-nitrophenyl]methanesulfonamide; N-[6-(2,4-difluorophenoxy)-2,3-dihydro-1-oxo-1H-inden-5-yl]methanesulfonamide; 3-(4-chlorophenoxy)-4-[(methylsulfonyl)amino]benzenesulfonamide; 3-(4-fluorophenoxy)-4-[(methylsulfonyl)amino]benzenesulfonamide; 3-[(1-methyl-1H-imidazol-2-yl)thio]-4 [(methylsulfonyl)amino]benzenesulfonamide; 5,5-dimethyl-4-[4-(methylsulfonyl)phenyl]-3-phenoxy-2(5H)-furanone; N-[6-[(4-ethyl-2-thiazolyl)thio]-1,3-dihydro-1-oxo-5-isobenzofuranyl]methanesulfonamide; 3-[(2,4-dichlorophenyl)thio]-4-[(methylsulfonyl)amino]benzene-sulfonamide; N-(2,3-dihydro-1,1-dioxido-6-phenoxy-1,2-benzisothiazol-5-yl)methanesulfonamide; N-[3-(formylamino)-4-oxo-6-phenoxy-4H-1-benzopyran-7-yl]methanesulfonamide; and 5-ethyl-2-(2′,4′-dichloro-6′-methylanilino)phenylacetic acid.

Essentially any cachexic condition will be treatable with the method of the present condition. For example, without limitation, the cachexic condition can be caused by cancer, hypophysiopriva, infectious disease, a cardiovascular condition (e.g., heart failure), anorexia, and others.

For the purposes of the present invention, a COX-2 selective inhibitor is any compound which inhibits COX-2 more than it inhibits COX-1. In other words, such a compound has a lower IC₅₀ for COX-2 than it has for COX-1. Preferably, the COX-2 selective inhibitor has a COX-1 IC₅₀:COX-2 IC₅₀ ratio of at least 2, more preferably at least 10, still more preferably at least 50, and more preferably still at least 100.

A class of selective cyclooxygenase-2 inhibiting agents useful in the methods, combinations and compositions of the present invention include compounds of Formula 1:

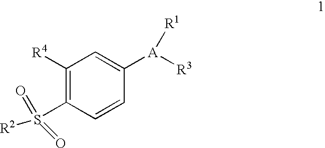

wherein

A is a 5- or 6-member ring substituent selected from aryl, heteroaryl, heterocyclo, and cycloalkyl, wherein A is optionally substituted with one or more radicals selected from hydroxy, alkyl, halo, oxo, and alkoxy;

R¹ is cyclohexyl, pyridinyl, or phenyl, wherein R¹ is optionally substituted with one or more radicals selected from alkyl, haloalkyl, cyano, carboxyl, alkoxycarbonyl, hydroxyl, hydroxyalkyl, haloalkoxy, amino, alkylamino, phenylamino, nitro, alkoxyalkyl, alkylsulfinyl, halo, alkoxy, and alkylthio;

R² is alkyl or amino;

R³ is selected from the group consisting of halo, alkyl, alkenyl, alkynyl, aryl, heteroaryl, oxo, cyano, carboxyl, cyanoalkyl, heterocyclyloxy, alkyloxy, alkylthio, alkylcarbonyl, cycloalkyl, phenyl, haloalkyl, heterocyclo, cycloalkenyl, phenylalkyl, heterocycloalkyl, alkylthioalkyl, hydroxyalkyl, alkoxycarbonyl, phenylcarbonyl, phenylalkylcarbonyl, phenylalkenyl, alkoxyalkyl, phenylthioalkyl, phenyloxyalkyl, alkoxyphenylalkoxyalkyl, alkoxycarbonylalkyl, aminocarbonyl, aminocarbonylalkyl, alkylaminocarbonyl, N-phenylaminocarbonyl, N-alkyl-N-phenylaminocarbonyl, alkylaminocarbonylalkyl, carboxyalkyl, alkylamino, N-arylamino, N-arylkylamino, N-alkyl-N-arylkylamino, N-alkyl-N-arylamino, aminoalkyl, alkylaminoalkyl, N-phenylaminoalkyl, N-phenylalkylaminoalkyl, N-alkyl-N-phenylalkylaminoalkyl, N-alkyl-N-phenylaminoalkyl, phenyloxy, phenylalkoxy, phenylthio, phenylalkylthio, alkylsulfinyl, alkylsulfonyl, aminosulfonyl, alkylaminosulfonyl, N-phenylaminosulfonyl, phenylsulfonyl, and N-alkyl-N-phenylaminosulfonyl; and

R⁴ is hydrido or halo;

or an isomer, tautomer, pharmaceutically-acceptable salt or prodrug thereof.

Within Formula 1 there is a subclass of compounds of particular interest wherein A is thienyl, oxazolyl, furyl, furanone, pyrrolyl, thiazolyl, imidazolyl, benzofuryl, indenyl, benzithienyl, isoxazolyl, pyrazolyl, cyclopentenyl, cyclopentadienyl, benzindazolyl, cyclopentenone, benzopyranopyrazolyl, phenyl, or pyridyl;

R¹ is cyclohexyl, pyridinyl, and phenyl, wherein cyclohexyl, pyridinyl, or phenyl, wherein R¹ is optionally substituted with one or more radicals selected from alkyl, haloalkyl, cyano, carboxyl, alkoxycarbonyl, hydroxyl, hydroxyalkyl, haloalkoxy, amino, alkylamino, phenylamino, nitro, alkoxyalkyl, alkylsulfinyl, alkoxy, halo, alkoxy, and alkylthio;

R² is methyl or amino; and

R³ is halo, alkyl, alkenyl, alkynyl, aryl, heteroaryl, oxo, cyano, carboxyl, cyanoalkyl, heterocyclyloxy, alkyloxy, alkylthio, alkylcarbonyl, cycloalkyl, phenyl, haloalkyl, heterocyclo, cycloalkenyl, phenylalkyl, heterocyclylalkyl, alkylthioalkyl, hydroxyalkyl, alkoxycarbonyl, phenylcarbonyl, phenylalkylcarbonyl, phenylalkenyl, alkoxyalkyl, phenylthioalkyl, phenylyloxyalkyl, alkoxyphenylalkoxyalkyl, alkoxycarbonylalkyl, aminocarbonyl, aminocarbonylalkyl, alkylaminocarbonyl, N-phenylaminocarbonyl, N-alkyl-N-phenylaminocarbonyl, alkylaminocarbonyl-alkyl, carboxy-alkyl, alkylamino, N-arylamino, N-arylkylamino, N-alkyl-N-arylkylamino, N-alkyl-N-arylamino, amino-alkyl, alkylaminoalkyl, N-phenylamino-alkyl, N-phenyl-alkylaminoalkyl, N-alkyl-N-phenyl-alkylamino-alkyl, N-alkyl-N-phenylaminoalkyl, phenyloxy, phenylalkoxy, phenylthio, phenylalkylthio, alkylsulfinyl, alkylsulfonyl, aminosulfonyl, alkylaminosulfonyl, N-phenylaminosulfonyl, phenylsulfonyl, or N-alkyl-N-phenylaminosulfonyl;

or an isomer, tautomer, pharmaceutically-acceptable salt or prodrug thereof.

Another class of compounds within Formula 1 of even more interest include compounds wherein A is substituted with one or more radicals selected alkyl, halo, oxo, and alkoxy;

R¹ is pyridyl, cyclohexyl, or phenyl, wherein R¹ is optionally substituted with one or more radicals selected from alkyl, halo, and alkoxy;

R³ is halo, alkyl, cyano, carboxyl, alkyloxy, phenyl, haloalkyl, or hydroxyalkyl; and

R⁴ is hydrido or fluoro;

or an isomer, tautomer, pharmaceutically-acceptable salt or prodrug thereof.

Some individual compounds within Formula 1, each of which are of particular interest in the present invention include compounds and pharmaceutically-acceptable salts thereof, as follows:

a. 4-(4-(methylsulfonyl)phenyl]-3 -phenyl-2(5H)-furanone (rofecoxib).

b. 4-[5-(4-methylphenyl)-3-(trifluoromethyl)-1H-pyrazol-1-yl]-benzenesulfonamide (celecoxib).

c. 4-[5-methyl-3-phenyl-3-phenylisoxazol-4-yl]benzenesulfonamide (valdecoxib).

d. 4-[5-(3-fluoro-4mthoxyphenyl)-3-difluoromethyl)-1H-pyrazol-1-yl]benzenesulfonamide (deracoxib).

e. 4-(4-cyclohexyl-2-methyloxazol-5-yl)-2-fluorobenzenesulfonamide (JTE-522).

f. 2-(6-methylpyrid-3-yl)-3-(4-methylsulfinylphenyl)-5-chloropyridine (etoricoxib).

g. 5-chloro-3-(4-(methylsulfonyl)phenyl)-2-(methyl-5-pyridinyl)pyridine.

h. 2-(3,5-difluorophenyl)-3-4-(methylsulfonyl)phenyl)-2-cyclopenten-1-one.

i. N-[[4-(5-methyl-3-phenylisoxazol-4yl]phenyl]sulfonyl]propanamide.

j. 4-[5-(4-chorophenyl)-3-(trifluoromethyl)-1H-pyrazole-1-yl]benzenesulfonamide.

k. 3-(3,4-difluorophenoxy)-5,5-dimethyl-4-[4-(methylsulfonyl)phenyl]-2(5H)-furanone.

l. N-[6-[(2,4-difluorophenyl)thio]-2,3-dihydro-1-oxo-1H-inden-5-yl]methanesulfonamide.

m. 3-(4-chlorophenyl)-4-[4-(methylsulfonyl)phenyl]-2(3H)-oxazolone.

n. 4-[3-(4-fluorophenyl)-2,3-dihydro-2-oxo-4-oxazolyl]benzenesulfonamide.

o. 3-[4-(methylsulfonyl)phenyl]-2-phenyl-2-cyclopenten-1-one.

p. 4-(2-methyl-4-phenyl-5-oxazolyl)benzenesulfonamide.

q. 3-(4-fluorophenyl)-4-[4-(methylsulfonyl)phenyl]-2(3H)-oxazolone.

r. 5-(4-fluorophenyl)-1-[4-(methylsulfonyl)phenyl]-3-(trifluoromethyl)-1H-pyrazole.

s. 4-[5-phenyl)-3-(trifluoromethyl)-1H-pyrazol-1-yl)benzenesulfonamide.

t. 4-[1-phenyl-3-(trifluoromethyl)-1H-pyrazol-5-yl]benzenesulfonamide.

u. 4-[5-(4-fluorophenyl)-3-(trifluoromethyl)-1H-pyrazol-1-yl]benzenesulfonamide.

v. N-[2-(cyclohexyloxy)-4-nitrophenyl]methanesulfonamide.

w. N-[6-(2,4-difluorophenoxy)-2,3-dihydro-1-oxo-1H-inden-5-yl]methanesulfonamide.

x. 3-(4-chlorophenoxy)-4-[(methylsulfonyl)amino]benzenesulfonamide.

y. 3-(4-fluorophenoxy)-4-[(methylsulfonyl)amino]benzenesulfonamide.

z. 3 -[(1-methyl-1H-imidazol-2-yl)thio]-4[methylsulfonyl) amino]benzenesulfonamide.

aa. 5,5-dimethyl-4-[4-(methylsulfonyl)phenyl]-3-phenoxy-2(5H)-furanone.

ab. N-[6-[(4-ethyl-2-thiazolyl)thio]-1,3-dihydro-1-oxo-5-isobenzofuranyl]methanesulfonamide.

ac. 3-[(2,4-dichlorophenyl)thio]-4-[(methylsulfonyl)amino]benzenesulfonamide.

ad. 1-fluoro-4-[2-[4-(methylsulfonyl)phenyl]cyclopenten-1-yl]benzene.

ae. 4-[5-(4-chlorophenyl)-3-(difluoromethyl)-1H-pyrazol-1-yl]benzenesulfonamide.

af. 3-[1-[4-(methylsulfonyl)phenyl]-4-(trifluoromethyl)-1H-imidazol-2-yl]pyridine.

ag. 4-[2-(3-pyridinyll)-4-(trifluoromethyl)-1H-imidazol-1-yl]benzenesulfonamide.

ah. 4-[5-(hydroxymethyl)-3-phenylisoxazol-4-yl]benzenesulfonamide.

ai. 4-[3-(4-chlorophenyl)-2,3-dihydro-2-oxo-4-oxazolyl]benzenesulfonamide.

aj. 4-[5-(difluoromethyl)-3-phenylisoxazol-4-yl]benzenesulfonamide.

ak. [1,1′,2′,1″-terphenyl]-4-sulfonamide.

al. 4-(methylsulfonyl)-1,1′,2],1″-terphenyl.

am. 4-(2-phenyl-3-pyridinyl)benzenesulfonamide.

an. N-(2,3-dihydro-1,1-dioxido-6-phenoxy-1,2-benzisothiazol-5-yl)methanesulfonamide.

ao. N-[3-(formylamino)-4-oxo-6-phenoxy-4H-1-benzopyran-7-yl]methanesulfonamide.

ap. 6-[[5-(4-chlorobenzoyl)-1,4-dimethyl-1H-pyrrol-2-yl]methyl]-3(2H)-pyridazinone.

aq. N-(4-nitro-2-phenoxyphenyl)methanesulfonamide.

Another class of selective cyclooxygenase-2 inhibiting agents useful in the methods and compositions of the present invention include substituted benzopyran cyclooxygenase-2 inhibiting compounds (also known as chromenes). Some chromene compounds useful in the present invention can be represented by the structure of Formula 2:

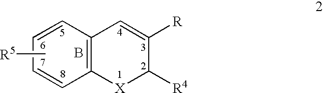

wherein

X is O, S or NR^{a};

R^{a} is alkyl;

R is carboxyl, alkyl, aralkyl, aminocarbonyl, alkylsulfonylaminocarbonyl or alkoxycarbonyl;

R⁴ is haloalkyl, alkyl, aralkyl, cycloalkyl or aryl, wherein aryl is optionally substituted with one or more radicals selected from alkylthio, nitro and alkylsulfonyl; and

R⁵ is one or more radicals independently selected from hydrido, halo, alkyl, aralkyl, alkoxy, aryloxy, heteroaryloxy, aralkyloxy, heteroaralkyloxy, haloalkyl, haloalkoxy, alkylamino, arylamino, aralkylamino, heteroarylamino, heteroarylalkylamino, nitro, amino, aminosulfonyl, alkylaminosulfonyl, arylaminosulfonyl, heteroarylaminosulfonyl, aralkylaminosulfonyl, heteroaralkylaminosulfonyl, heterocyclosulfonyl, alkylsulfonyl, optionally substituted aryl, optionally substituted heteroaryl, aralkylcarbonyl, heteroarylcarbonyl, arylcarbonyl, aminocarbonyl, and alkylcarbonyl;

or R⁵ together with ring B forms a naphthyl radical;

or an isomer, tautomer, pharmaceutically-acceptable salt or prodrug thereof.

Within Formula 2 there is a subclass of compounds of particular interest wherein

X is O or S;

R is carboxyl, lower alkyl, lower aralkyl or lower alkoxycarbonyl;

R⁴ is lower haloalkyl, lower cycloalkyl or phenyl; and

R⁵ is one or more radicals independently selected from hydrido, halo, lower alkyl, lower alkoxy, lower haloalkyl, lower haloalkoxy, lower alkylamino, nitro, amino, aminosulfonyl, lower alkylaminosulfonyl, 5- or 6-membered heteroarylalkylaminosulfonyl, lower aralkylaminosulfonyl, 5- or 6-membered nitrogen containing heterocyclosulfonyl, lower alkylsulfonyl, optionally substituted phenyl, lower aralkylcarbonyl, and lower alkylcarbonyl;

or an isomer, tautomer, pharmaceutically-acceptable salt or prodrug thereof.

Another class of compounds within Formula 2 of interest include compounds wherein

R is carboxyl;

R⁴ is lower haloalkyl; and

R⁵is one or more radicals independently selected from hydrido, halo, lower alkyl, lower haloalkyl, lower haloalkoxy, lower alkylamino, amino, aminosulfonyl, lower alkylaminosulfonyl, 5- or 6-membered heteroarylalkylaminosulfonyl, lower aralkylaminosulfonyl, lower alkylsulfonyl, 6-membered nitrogen containing heterocyclosulfonyl, optionally substituted phenyl, lower aralkylcarbonyl, and lower alkylcarbonyl;

or an isomer, tautomer, pharmaceutically-acceptable salt or prodrug thereof.

Still another class of compounds within Formula 2 of interest include compounds wherein

R⁴ is fluoromethyl, chloromethyl, dichloromethyl, trichloromethyl, pentafluoroethyl, heptafluoropropyl, difluoroethyl, difluoropropyl, dichloroethyl, dichloropropyl, difluoromethyl, or trifluoromethyl; and

R⁵ is one or more radicals independently selected from hydrido, chloro, fluoro, bromo, iodo, methyl, ethyl, isopropyl, tert-butyl, butyl, isobutyl, pentyl, hexyl, methoxy, ethoxy, isopropyloxy, tertbutyloxy, trifluoromethyl, difluoromethyl, trifluoromethoxy, amino, N,N-dimethylamino, N,N-diethylamino, N-phenylmethylaminosulfonyl, N-phenylethylaminosulfonyl, N-(2-furylmethyl)aminosulfonyl, nitro, N,N-dimethylaminosulfonyl, aminosulfonyl, N-methylaminosulfonyl, N-ethylsulfonyl, 2,2-dimethylethylaminosulfonyl, N,N-dimethylaminosulfonyl, N-(2-methylpropyl)aminosulfonyl, N-morpholinosulfonyl, methylsulfonyl, benzylcarbonyl, 2,2-dimethylpropylcarbonyl, phenylacetyl and phenyl;

or an isomer, tautomer, pharmaceutically-acceptable salt or prodrug thereof.

An additional class of compounds within Formula 2 of interest include compounds wherein

R is carboxyl;

R⁴ is trifluoromethyl or pentafluorethyl; and

R⁵ is one or more radicals independently selected from hydrido, chloro, fluoro, bromo, iodo, methyl, ethyl, isopropyl, tert-butyl, methoxy, trifluoromethyl, trifluoromethoxy, N-phenylmethylaminosulfonyl, N-phenylethylaminosulfonyl, N-(2-furylmethyl)aminosulfonyl, N,N-dimethylaminosulfonyl, N-methylaminosulfonyl, N-(2,2-dimethylethyl)aminosulfonyl, dimethylaminosulfonyl, 2-methylpropylaminosulfonyl, N-morpholinosulfonyl, methylsulfonyl, benzylcarbonyl, and phenyl;

or an isomer, tautomer, pharmaceutically-acceptable salt or prodrug thereof.

A family of specific compounds within Formula 2 of particular interest include compounds and their isomers and pharmaceutically-acceptable salts thereof, as follows:

6-chloro-2-trifluoromethyl-2H-1-benzopyran-3-carboxylic acid,

6-chloro-7-methyl-2-trifluoromethyl-2H-1-benzopyran-3-carboxylic acid,

8-(1-methylethyl)-2-trifluoromethyl-2H-1-benzopyran-3-carboxylic acid,

6-chloro-7-(1,1-dimethylethyl)-2-trifluoromethyl-2H-1-benzopyran-3-carboxylic acid,

6-chloro-8-(1-methylethyl)-2-trifluoromethyl-2H-1-benzopyran-3-carboxylic acid,

2-trifluoromethyl-3H-naphthopyran-3-carboxylic acid,

7-(1,1-dimethylethyl)-2-trifluoromethyl-2H-1-benzopyran-3-carboxylic acid,

6-bromo-2-trifluoromethyl-2H-1-benzopyran-3-carboxylic acid,

8-chloro-2-trifluoromethyl-2H-1-benzopyran-3-carboxylic acid,

6-trifluoromethoxy-2-trifluoromethyl-2H-1-benzopyran-3-carboxylic acid,

5,7-dichloro-2-trifluoromethyl-2H-1-benzopyran-3-carboxylic acid,

8-phenyl-2-trifluoromethyl-2H-1-benzopyran-3-carboxylic acid,

7,8-dimethyl-2-trifluoromethyl-2H-1-benzopyran-3-carboxylic acid,

6,8-bis(dimethylethyl)-2-trifluoromethyl-2H-1-benzopyran-3-carboxylic acid,

7-(1-methylethyl)-2-trifluoromethyl-2H-1-benzopyran-3-carboxylic acid,

7-phenyl-2-trifluoromethyl-2H-1-benzopyran-3-carboxylic acid,

6-chloro-7-ethyl-2-trifluoromethyl-2H-1-benzopyran-3-carboxylic acid,

6-chloro-8-ethyl-2-trifluoromethyl-2H-1-benzopyran-3-carboxylic acid,

6-chloro-7-phenyl-2-trifluoromethyl-2H-1-benzopyran-3-carboxylic acid,

6,7-dichloro-2-trifluoromethyl-2H-1-benzopyran-3-carboxylic acid,

6,8-dichloro-2-trifluoromethyl-2H-1-benzopyran-3-carboxylic acid,

2-trifluoromethyl-3H-naptho[2,1-b]pyran-3-carboxylic acid,

6-chloro-8-methyl-2-trifluoromethyl-2H-1-benzopyran-3-carboxylic acid,

8-chloro-6-methyl-2-trifluoromethyl-2H-1-benzopyran-3-carboxylic acid,

8-chloro-6-methoxy-2-trifluoromethyl-2H-1-benzopyran-3-carboxylic acid,

6-bromo-8-chloro-2-trifluoromethyl-2H-1-benzopyran-3-carboxylic acid,

8-bromo-6-fluoro-2-trifluoromethyl-2H-1-benzopyran-3-carboxylic acid,

8-bromo-6-methyl-2-trifluoromethyl-2H-1-benzopyran-3-carboxylic acid,

8-bromo-5-fluoro-2-trifluoromethyl-2H-1-benzopyran-3-carboxylic acid,

6-chloro-8-fluoro-2-trifluoromethyl-2H-1-benzopyran-3-carboxylic acid,

6-bromo-8-methoxy-2-trifluoromethyl-2H-1-benzopyran-3-carboxylic acid,

6-[[(phenylmethyl)amino]sulfonyl]-2-trifluoromethyl-2H-1-benzopyran-3-carboxylic acid,

6-[(dimethylamino)sulfonyl]-2-trifluoromethyl-2H-1-benzopyran-3-carboxylic acid,

6-[(methylamino)sulfonyl]-2-trifluoromethyl-2H-1-benzopyran-3-carboxylic acid,

6-[(4-morpholino)sulfonyl]-2-trifluoromethyl-2H- 1-benzopyran-3-carboxylic acid,

6-[(1,1-dimethylethyl)aminosulfonyl]-2-trifluoromethyl-2H-1-benzopyran-3-carboxylic acid,

6-[(2-methylpropyl)aminosulfonyl]-2-trifluoromethyl-2H-1-benzopyran-3-carboxylic acid,

6-methylsulfonyl-2-trifluoromethyl-2H-1-benzopyran-3-carboxylic acid,

8-chloro-6-[[(phenylmethyl)amino]sulfonyl]-2-trifluoromethyl-2H-1-benzopyran-3-carboxylic acid,

6-phenylacetyl-2-trifluoromethyl-2H-1-benzopyran-3-carboxylic acid,

6,8-dibromo-2-trifluoromethyl-2H-1-benzopyran-3-carboxylic acid,

8-chloro-5,6-dimethyl-2-trifluoromethyl-2H-1-benzopyran-3-carboxylic acid,

6,8-dichloro-(S)-2-trifluoromethyl-2H-1-benzopyran-3-carboxylic acid,

6-benzylsulfonyl-2-trifluoromethyl-2H-1-benzopyran-3-carboxylic acid,

6-[[N-(2-furylmethyl)amino]sulfonyl]-2-trifluoromethyl-2H-1-benzopyran-3-carboxylic acid,

6-[[N-(2-phenylethyl)amino]sulfonyl]-2-trifluoromethyl-2H-1-benzopyran-3-carboxylic acid,

6-iodo-2-trifluoromethyl-2H-1-benzopyran-3-carboxylic acid,

7-(1,1-dimethylethyl)-2-pentafluoroethyl-2H-1-benzopyran-3-carboxylic acid, and

6-chloro-2-trifluoromethyl-2H-1-benzothiopyran-3-carboxylic acid.

Another class of selective cyclooxygenase-2 inhibiting agents useful in the methods, combinations and compositions of the present invention include compounds of Formula 3:

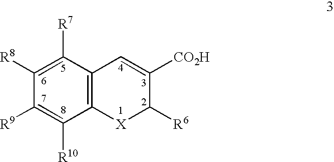

wherein

X is O, S or NR^{a};

R^{a} is alkyl;

R⁶ is lower haloalkyl;

R⁷ is hydrido or halo;

R⁸ is hydrido, halo, lower alkyl, lower haloalkoxy, lower alkoxy, lower aralkylcarbonyl, lower dialkylaminosulfonyl, lower alkylaminosulfonyl, lower aralkylaminosulfonyl, lower heteroaralkylaminosulfonyl, or 5- or 6-membered nitrogen containing heterocyclosulfonyl;

R⁹ is hydrido, lower alkyl, halo, lower alkoxy, or aryl; and

R¹⁰ is hydrido, halo, lower alkyl, lower alkoxy, or aryl;

or an isomer or pharmaceutically-acceptable salt or prodrug thereof.

Within Formula 3 there is a subclass of compounds of particular interest wherein

R⁶ is trifluoromethyl or pentafluoroethyl;

R⁷ is hydrido, chloro, or fluoro;

R⁸ is hydrido, chloro, bromo, fluoro, iodo, methyl, tert-butyl, trifluoromethoxy, methoxy, benzylcarbonyl, dimethylaminosulfonyl, isopropylaminosulfonyl, methylaminosulfonyl, benzylaminosulfonyl, phenylethylaminosulfonyl, methylpropylaminosulfonyl, methylsulfonyl, or morpholinosulfonyl;

R⁹ is hydrido, methyl, ethyl, isopropyl, tert-butyl, chloro, methoxy, diethylamino, or phenyl; and

R¹⁰ is hydrido, chloro, bromo, fluoro, methyl, ethyl, tert-butyl, methoxy, or phenyl;

or an isomer, tautomer, pharmaceutically-acceptable salt or prodrug thereof.

Specific compounds of interest within Formula 3 include each of the compounds and pharmaceutically-acceptable salts thereof as follows:

6-Chloro-2-trifluoromethyl-2H-1-benzopyran-3-carboxylic acid,

(S)-6-Chloro-2-trifluoromethyl-2H-1-benzopyran-3-carboxylic acid,

6-Chloro-7-(1,1 -dimethylethyl)-2-trifluoromethyl-2H-1-benzopyran-3-carboxylic acid,

(S)-6-Chloro-7-(1,1-dimethylethyl)-2-(trifluoromethyl)-2H-1-benzopyran-3-carboxylic acid,

6-Trifluoromethoxy-2-trifluoromethyl-2H-1-benzopyran-3-carboxylic acid,

(S)-6-Trifluoromethoxy-2-trifluoromethyi-2H-1-benzopyran-3-carboxylic acid,

6-Formyl-2-(trifluoromethyl)-2H-1-benzopyran-3-carboxylic acid,

6-(Difluoromethyl)-2-(trifluoromethyl)-2H-1-benzopyran-3-carboxylic acid,

6,8-Dichloro-7-methyl-2-(trifluoromethyl)-2H-1-benzopyran-3-carboxylic acid,

6,8-Dichloro-2-trifluoromethyl-2H-1-benzopyran-3-carboxylic acid,

(S)-6,8-Dichloro-2-(trifluoromethyl)-2H-1-benzopyran-3-carboxylic acid,

6-Chloro-1,2-dihydro-2-(trifluoromethyl)-3-quinolinecarboxylic acid,

(S)-6-chloro-1,2-dihydro-2-(trifluoromethyl)-3-quinolinecarboxylic acid,

6,8-Dichloro-1,2-dihydro-2-(trifluoromethyl)-3-quinolinecarboxylic acid,

7-(1,1-Dimethylethyl)-2-trifluoromethyl-2H-1-benzopyran-3-carboxylic acid,

6,7-Dichloro-2-trifluoromethyl-2H-1-benzopyran-3-carboxylic acid,

5,6-Dichloro-2-(trifluoromethyl)-2H-1-benzopyran-3-carboxylic acid,

2,6-Bis(trifluoromethyl)-2H-1-benzopyran-3-carboxylic acid,

5,6,7-Trichloro-2-(trifluoromethyl)-2H-1-benzopyran-3-carboxylic acid,

6,7,8-Trichloro-2-(trifluoromethyl)-2H-1-benzopyran-3-carboxylic acid,

6-Iodo-1,2-dihydro-2-(trifluoromethyl)-3-quinolinecarboxylic acid,

6-Bromo-1,2-dihydro-2-(trifluoromethyl)-3-quinolinecarboxylic acid,

6-Chloro-7-methyl-2-(trifluoromethyl)-2H-1-benzothiopyran-3-carboxylic acid, and

6,8-Dichloro-2-trifluoromethyl-2H-1-benzothiopyran-3-carboxylic acid.

Specific compounds of particular interest within Formula 3 include each of the compounds and pharmaceutically-acceptable salts thereof as follows:

6-Chloro-2-trifluoromethyl-2H-1-benzopyran-3-carboxylic acid,

(S)-6-Chloro-2-trifluoromethyl-2H-1-benzopyran-3-carboxylic acid,

6-Chloro-7-(1,1-dimethylethyl)-2-trifluoromethyl-2H-1-benzopyran-3-carboxylic acid,

(S)-6-Chloro-7-(1,1-dimethylethyl)-2-(trifluoromethyl)-2H-1-benzopyran-3-carboxylic acid,

6-Trifluoromethoxy-2-trifluoromethyl-2H-1-benzopyran-3-carboxylic acid,

(S)-6-Trifluoromethoxy-2-trifluoromethyl-2H-1-benzopyran-3-carboxylic acid,

6-Formyl-2-(trifluoromethyl)-2H-1-benzopyran-3-carboxylic acid,

6-(Difluoromethyl)-2-(trifluoromethyl)-2H-1-benzopyran-3-carboxylic acid,

6,8-Dichloro-7-methyl-2-(trifluoromethyl)-2H-1-benzopyran-3-carboxylic acid,

6,8-Dichloro-2-trifluoromethyl-2H-1-benzopyran-3-carboxylic acid,

(S)-6,8-Dichloro-2-(trifluoromethyl)-2H-1-benzopyran-3-carboxylic acid,

6-Chloro-1,2-dihydro-2-(trifluoromethyl)-3-quinolinecarboxylic acid,

(S)-6-chloro-1,2-dihydro-2-(trifluoromethyl)-3-quinolinecarboxylic acid, and

6,8-Dichloro-1,2-dihydro-2-(trifluoromethyl)-3-quinolinecarboxylic acid.

Other individual selective cyclooxygenease-2 inhibiting agents useful in the methods and compositions of the present invention include compounds and pharmaceutically-acceptable salts thereof as follows:

5-ethyl-2-(2′,4′-dichloro-6′-methylanilino)phenylacetic acid;

5-ethyl-2-(2′,3′,5′,6′-tetrafluoroanilino)phenylacetic acid;

5-methyl-2-(2′,4′-difluoro-6′-chloroanilino)phenylacetic acid;

5-methyl-2-(2′-fluoro-6′-chloroanilino)phenylacetic acid;

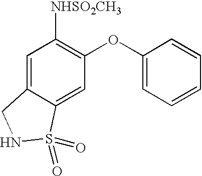

N-(2,3 -dihydro-1,1-dioxido-6-phenoxy-1,2-benzisothiazol-5-yl)methanesulfonamide;

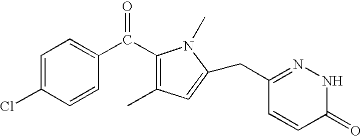

6-[[5-(4-chlorobenzoyl)-1,4-dimethyl-1H-pyrrol-2-yl]methyl]-3(2H)-pyridazinone;

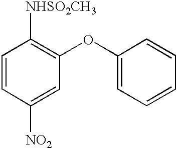

N-(4-nitro-2-phenoxyphenyl)methanesulfonamide;

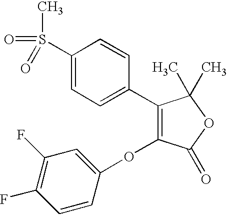

3-(3,4-difluorophenoxy)-5,5-dimethyl-4-[4-(methylsulfonyl)phenyl]-2(5H)-furanone;

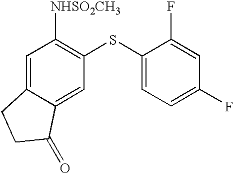

N-[6-[(2,4-difluorophenyl)thio]-2,3-dihydro-1-oxo-1H-inden-5-yl]methanesulfonamide;

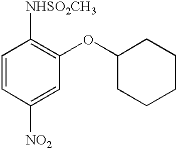

N-[2-(cyclohexyloxy)-4-nitrophenyl]methanesulfonamide;

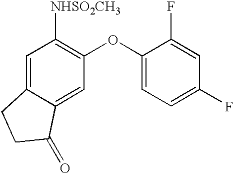

N-[6-(2,4-difluorophenoxy)-2,3-dihydro-1-oxo-1H-inden-5-yl]methanesulfonamide;

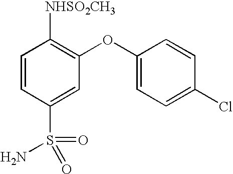

3-(4-chlorophenoxy)-4-[(methylsulfonyl)amino]benzenesulfonamide;

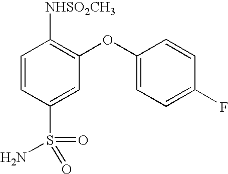

3-(4-fluorophenoxy)-4-[(methylsulfonyl)amino]benzenesulfonamide;

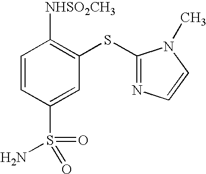

3-[(1-methyl-1H-imidazol-2-yl)thio]-4[(methylsulfonyl) amino]benzenesulfonamide;

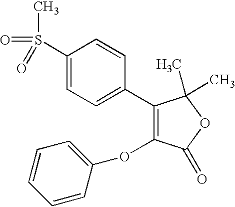

5,5-dimethyl-4-[4-(methylsulfonyl)phenyl]-3-phenoxy-2(5H)-furanone;

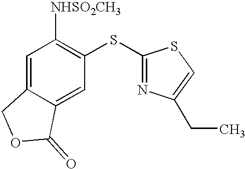

N-[6-[(4-ethyl-2-thiazolyl)thio]-1,3-dihydro-1-oxo-5-isobenzofuranyl]methanesulfonamide;

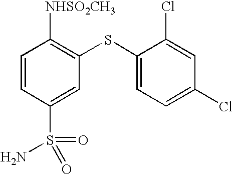

3-[(2,4-dichlorophenyl)thio]-4-[(methylsulfonyl)amino]benzenesulfonamide;

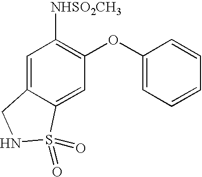

N-(2,3-dihydro-1,1-dioxido-6-phenoxy-1,2-benzisothiazol-5-yl)methanesulfonamide; and

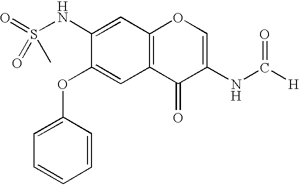

N-[3-(formylamino)-4-oxo-6-phenoxy-4H-1-benzopyran-7-yl]methanesulfonamide.

In a yet further embodiment of the invention, the cyclooxygenase inhibitor used in connection with the methods of the present invention can be selected from the class of phenylacetic acid derivative cyclooxygenase-2 selective inhibitors represented by the general structure of Formula 4:

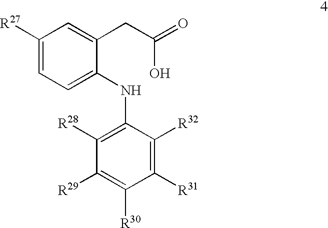

or an isomer, a pharmaceutically acceptable salt, ester, or prodrug thereof; wherein:

R²⁷ is methyl, ethyl, or propyl;

R²⁸ is chloro or fluoro;

R²⁹ is hydrogen, fluoro, or methyl;

R³⁰ is hydrogen, fluoro, chloro, methyl, ethyl, methoxy, ethoxy or hydroxy;

R³¹ is hydrogen, fluoro, or methyl; and

R³² is chloro, fluoro, trifluoromethyl, methyl, or ethyl, provided that R²⁸, R²⁹, R³⁰ and R³¹ are not all fluoro when R²⁷ is ethyl and R³⁰ is H.

Another suitable phenylacetic acid derivative cyclooxygenase-2 selective inhibitor that is described in WO 02/20090 is a compound that is referred to as COX-189 (also termed lumiracoxib), having CAS Reg. No. 220991-20-8, and having the structure shown in Formula 4, wherein:

R²⁷ is methyl;

R²⁸ is fluoro;

R³² is chloro; and

R²⁹, and R³¹ are hydrogen.

Compounds that have a structure similar to that shown in Formula 4, which can serve as the Cox-2 selective inhibitor of the present invention, are described in U.S. Pat. Nos. 6,310,099, 6,291,523, and 5,958,978.

In the written descriptions of molecules and groups, molecular descriptors can be combined to produce words or phrases that describe structural groups or are combined to describe structural groups. Such descriptors are used in this document. Common illustrative examples include such terms as aralkyl (or arylalkyl), heteroaralkyl, heterocycloalkyl, cycloalkylalkyl, aralkoxyalkoxycarbonyl, and the like. A specific example of a compound encompassed with the latter descriptor aralkoxyalkoxycarbonyl is C₆H₅—CH₂—CH₂—O—CH₂—O—(C═O)— wherein C₆H₅— is phenyl. It is also to be noted that a structural group can have more than one descriptive word or phrase in the art, for example, heteroaryloxyalkylcarbonyl can also be termed heteroaryloxyalkanoyl. Such combinations are used herein in the description of the processes, compounds and compositions of this invention and further examples are described below. The following list is not intended to be exhaustive or drawn out but provide illustrative examples of words or phrases (terms) that are used herein.

As utilized herein, the term “alkyl”, alone or in combination, means a straight-chain or branched-chain alkyl radical containing one to about twelve carbon atoms, preferably one to about ten carbon atoms, and more preferably one to about six carbon atoms. Examples of such radicals include methyl, ethyl, n-propyl, isopropyl, n-butyl, isobutyl, sec-butyl, tert-butyl, pentyl, iso-amyl, hexyl, octyl, and the like.

The term “alkenyl”, alone or in combination, means a straight-chain or branched-chain hydrocarbon radical having one or more double bonds and containing two to about twenty carbon atoms preferably two to about twelve carbon atoms, and more preferably, two to about six carbon atoms. Examples of suitable alkenyl radicals include ethenyl (vinyl), 2-propenyl, 3-propenyl, allyl, 1,4-pentadienyl, 1,4-butadienyl, 1-butenyl, 2-butenyl, 3-butenyl, 4-methylbutenyl, decenyl, and the like. The term “alkenyl” embrace radicals having “cis” and “trans” orientations, or alternatively, “E” and “Z” orientations.

The term “alkynyl”, alone or in combination, means a straight-chain or branched-chain hydrocarbon radical having one or more triple bonds and containing two to about twelve carbon atoms, preferably two to about ten carbon atoms, and more preferably, two to about six carbon atoms. Examples of alkynyl radicals include ethynyl, 2-propynyl, 3-propynyl, decynyl, 1-butynyl, 2-butynyl, 3-butynyl, propargyl, and the like.

The term “acyl”, alone or in combination, means a radical provided by the residue after removal of hydroxyl from an organic acid. Examples of such acyl radicals include alkanoyl and aroyl radicals. Examples of such alkanoyl radicals include formyl, acetyl, propionyl, butyryl, isobutyryl, valeryl, isovaleryl, pivaloyl, hexanoyl, trifluoroacetyl, and the like.

The term “carbonyl” or “oxo”, alone or in combination, i.e., used with other terms, such as “alkoxycarbonyl”, means a —C(═O)— group wherein the remaining two bonds (valences) can be independently substituted. The term carbonyl is also intended to encompass a hydrated carbonyl group —C(OH)₂—.

The term “hydrido”, alone or in combination, means a single hydrogen atom (H). This hydrido radical may be attached, for example, to an oxygen atom to form a hydroxyl radical or two hydrido radicals may be attached to a carbon atom to form a methylene (—CH₂—) radical.

The term “halo”, alone or in combination, means halogen such as fluoride, chloride, bromide or iodide.

The term “haloalkyl”, alone or in combination, means an alkyl radical having the significance as defined above wherein one or more hydrogens are replaced with a halogen. Specifically embraced are monohaloalkyl, dihaloalkyl and polyhaloalkyl radicals. A monohaloalkyl radical, for one example, may have either an iodo, bromo, chloro or fluoro atom within the radical. Dihalo and polyhaloalkyl radicals may have two or more of the same halo atoms or a combination of different halo radicals.

More preferred haloalkoxy radicals are haloalkoxy radicals having one to six carbon atoms and one or more halo radicals. Examples of such haloalkyl radicals include chloromethyl, dichloromethyl, trichloromethyl, 1-bromoethyl, fluoromethyl, difluoromethyl, trifluoromethyl, 1,1,1-trifluoroethyl, pentafluoroethyl, heptafluoropropyl, difluorochloromethyl, dichlorofluoromethyl, difluoroethyl, difluoropropyl, dichloroethyl, dichloropropyl, and the like.

Examples of such radicals include fluoromethoxy, chloromethoxy, trifluoromethoxy, trifluoroethoxy, fluoroethoxy, fluoropropoxy, and the like.

The term “perfluoroalkyl”, alone or in combination, means an alkyl group wherein each hydrogen has been replaced by a fluorine atom. Examples of such perfluoroalkyl groups, in addition to trifluoromethyl above, are perfluorobutyl, perfluoroisopropyl, perfluorododecyl and perfluorodecyl.

The term “perfluoroalkoxy”, alone or in combination, means a perfluoroalkyl ether radical wherein the term perfluoroalkyl is as defined above. Examples of such perfluoroalkoxy groups, in addition to trifluoromethoxy (F₃C—O—), are perfluorobutoxy, perfluoroisopropoxy, perfluorododecoxy and perfluorodecoxy.

The term “perfluoroalkylthio”, alone or in combination, means a perfluoroalkyl thioether radical wherein the term perfluoroalkyl is as defined above. Examples of such perfluoroalkylthio groups, in addition to trifluoromethylthio (F₃C—S—), are perfluorobutylthio, perfluoroisopropylthio, perfluorododecylthio and perfluorodecylthio.

The term “hydroxyalkyl”, alone or in combination, means a linear or branched alkyl radical having one to about ten carbon atoms any one of which may be substituted with one or more hydroxyl radicals. Preferred hydroxyalkyl radicals have one to six carbon atoms and one or more hydroxyl radicals. Examples of such radicals include hydroxymethyl, hydroxyethyl, hydroxypropyl, hydroxybutyl and hydroxyhexyl.

The term “thiol” or “sulfhydryl”, alone or in combination, means a —SH group. The term “thio” or “thia”, alone or in combination, means a thiaether group; i.e., an ether group wherein the ether oxygen is replaced by a sulfur atom.

The term “amino”, alone or in combination, means an amine or —NH₂ group whereas the term mono-substituted amino, alone or in combination, means a substituted amine —N(H)(substituent) group wherein one hydrogen atom is replaced with a substituent, and disubstituted amine means a —N(substituent)₂ wherein two hydrogen atoms of the amino group are replaced with independently selected substituent groups.

Amines, amino groups and amides are compounds that can be designated as primary (I∘), secondary (II∘) or tertiary (III∘) or unsubstituted, mono-substituted or N,N-disubstituted depending on the degree of substitution of the amino nitrogen. Quaternary amine (ammonium)(IV∘) means a nitrogen with four substituents [—N⁺(substituent)₄] that is positively charged and accompanied by a counter ion, whereas N-oxide means one substituent is oxygen and the group is represented as [—N⁺(substituent)₃-O⁻]; i.e., the charges are internally compensated.

The term “cyano”, alone or in combination, means a —C-triple bond-N (—C≡N) group.

The term “azido”, alone or in combination, means a —N-triple bond-N (—N≡N) group.

The term “hydroxyl”, alone or in combination, means a —OH group.

The term “nitro”, alone or in combination, means a —NO₂ group.

The term “azo”, alone or in combination, means a —N═N— group wherein the bonds at the terminal positions can be independently substituted.

The term “hydrazino”, alone or in combination, means a —NH—NH— group wherein the depicted remaining two bonds (valences) can be independently substituted. The hydrogen atoms of the hydrazino group can be replaced, independently, with substituents and the nitrogen atoms can form acid addition salts or be quaternized.

The term “sulfonyl”, alone or in combination, i.e., linked to other terms such as alkylsulfonyl, means a —SO₂— group wherein the depicted remaining two bonds (valences) can be independently substituted.

The term “sulfoxido”, alone or in combination, means a —SO— group wherein the remaining two bonds (valences) can be independently substituted.

The term “sulfone”, alone or in combination, means a —SO₂— group wherein the depicted remaining two bonds (valences) can be independently substituted.

The term “sulfenamide”, alone or in combination, means a —SON═ group wherein the remaining three depicted bonds (valences) can be independently substituted.

The term “sulfide”, alone or in combination, means a —S— group wherein the remaining two bonds (valences) can be independently substituted.

The term “alkylthio”, alone or in combination, means a radical containing a linear or branched alkyl radical, of one to about ten carbon atoms attached to a divalent sulfur atom. More preferred alkylthio radicals are radicals having alkyl radicals of one to six carbon atoms. Examples of such alkylthio radicals are methylthio, ethylthio, propylthio, butylthio and hexylthio.

The term “alkylthioalkyl”, alone or in combination, means a radical containing an alkylthio radical attached through the divalent sulfur atom to an alkyl radical of one to about ten carbon atoms. More preferred alkylthioalkyl radicals are radicals having alkyl radicals of one to six carbon atoms. Examples of such alkylthioalkyl radicals include methylthiomethyl, methylthioethyl, ethylthioethyl, and ethylthiomethyl.

The term “alkylsulfinyl”, alone or in combination, means a radical containing a linear or branched alkyl radical, of one to ten carbon atoms, attached to a divalent —S(═O)— radical. More preferred alkylsulfinyl radicals are radicals having alkyl radicals of one to six carbon atoms. Examples of such alkylsulfinyl radicals include methylsulfinyl, ethylsulfinyl, butylsulfinyl and hexylsulfinyl.

The term “alkylsulfonyl”, alone or in combination, means an alkyl radical attached to a sulfonyl radical, where alkyl is defined as above. More preferred alkylsulfonyl radicals are alkylsulfonyl radicals having one to six carbon atoms. Examples of such alkylsulfonyl radicals include methylsulfonyl, ethylsulfonyl and propylsulfonyl. The “alkylsulfonyl” radicals may be further substituted with one or more halo atoms, such as fluoro, chloro or bromo, to provide haloalkylsulfonyl radicals.

The terms “sulfamyl”, “aminosulfonyl” and “sulfonamidyl”, alone or in combination, mean a NH₂O₂S— radical.

The term “alkoxy” or “alkyloxy”, alone or in combination, mean an alkyl ether radical wherein the term alkyl is as defined above. Examples of suitable alkyl ether radicals include methoxy, ethoxy, n-propoxy, isopropoxy, n-butoxy, iso-butoxy, sec-butoxy, tert-butoxy, and the like. The “alkoxy” radicals may be further substituted with one or more halo atoms, such as fluoro, chloro or bromo, to provide haloalkoxy radicals. More preferred haloalkoxy radicals are “haloalkoxy” radicals having one to six carbon atoms and one or more halo radicals. Examples of such radicals include fluoromethoxy, chloromethoxy, trifluoromethoxy, trifluoroethoxy, fluoroethoxy and fluoropropoxy.

The term “alkoxyalkyl”, alone or in combination, means an alkyl radical having one or more alkoxy radicals attached to the alkyl radical, that is, to form monoalkoxyalkyl and dialkoxyalkyl radicals. The “alkoxy” radicals may be further substituted with one or more halo atoms, such as fluoro, chloro or bromo, to provide haloalkoxy radicals.

The term “cycloalkyl”, alone or in combination, means a cyclic alkyl radical that contains three to about twelve carbon atoms. More preferred cycloalkyl radicals are cycloalkyl radicals having three to about eight carbon atoms. Examples of such radicals include cyclopropyl, cyclobutyl, cyclopentyl, cyclohexyl, and the like

The term “cycloalkylalkyl”, alone or in combination, means an alkyl radical as defined above that is substituted by a cycloalkyl radical containing three to about eight, preferably three to about six, carbon atoms. Examples of such cycloalkyl radicals include cyclopropyl, cyclobutyl, cyclopentyl, cyclohexyl, and the like.

The term “cycloalkenyl” means partially unsaturated carbocyclic radicals having three to twelve carbon atoms. More preferred cycloalkenyl radicals are cycloalkenyl radicals having four to about eight carbon atoms. Examples of such radicals include cyclobutenyl, cyclopentenyl, cyclohexenyl, and the like.

The term “heterocyclo” embraces saturated, partially unsaturated and unsaturated heteroatom-containing ring-shaped radicals, where the heteroatoms may be selected from nitrogen, sulfur and oxygen. Examples of saturated heterocyclo radicals include saturated three- to six-membered heteromonocylic group containing one to four nitrogen atoms (e.g. pyrrolidinyl, imidazolidinyl, piperidino, piperazinyl, etc.); saturated three- to six-membered heteromonocyclic group containing one to two oxygen atoms and one to three nitrogen atoms (e.g. morpholinyl, etc.); saturated three- to six-membered heteromonocyclic group containing one to two sulfur atoms and one to three nitrogen atoms (e.g., thiazolidinyl, etc.). Examples of partially unsaturated heterocyclo radicals include dihydrothiophene, dihydropyran, dihydrofuran and dihydrothiazole. A heterocyclic (heterocyclo) portion of a heterocyclocarbonyl, heterocyclooxy-carbonyl, heterocycloalkoxycarbonyl, or heterocycloalkyl group or the like is a saturated or partially unsaturated monocyclic, bicyclic or tricyclic heterocycle that contains one or more hetero atoms selected from nitrogen, oxygen and sulphur. Heterocyclo compounds include benzofused heterocyclic compounds such as benzo-1,4-dioxane. Such a moiety can be optionally substituted on one or more ring carbon atoms by halogen, hydroxy, hydroxycarbonyl, alkyl, alkoxy, oxo, and the like, and/or on a secondary nitrogen atom (i.e., —NH—) of the ring by alkyl, aralkoxycarbonyl, alkanoyl, aryl or arylalkyl or on a tertiary nitrogen atom (i.e., ═N—) by oxido and that is attached via a carbon atom. The tertiary nitrogen atom with three substituents can also attached to form a N-oxide [═N(O)—] group.

The term “heterocycloalkyl”, alone or in combination, means a saturated and partially unsaturated heterocyclo-substituted alkyl radical, such as pyrrolidinylmethyl, and heteroaryl-substituted alkyl, such as pyridylmethyl, quinolylmethyl, thienylmethyl, furylethyl, and quinolylethyl. The heteroaryl in said heteroaralkyl may be additionally substituted with halo, alkyl, alkoxy, halkoalkyl and haloalkoxy.

The term “aryl”, alone or in combination, means a five- or six-membered carbocyclic aromatic ring-containing moiety or a five- or six-membered carbocyclic aromatic system containing two or three rings wherein such rings are attached together in a pendent manner, or a fused ring system containing two or three rings that have all carbon atoms in the ring; i.e., a carbocyclic aryl radical. The term “aryl” embraces aromatic radicals such as phenyl, indenyl, naphthyl, tetrahydronaphthyl, indane and biphenyl. Aryl moieties may also be substituted with one or more substituents including alkyl, alkoxyalkyl, alkylaminoalkyl, carboxyalkyl, alkoxycarbonylalkyl, aminocarbonylalkyl, alkoxy, aralkoxy, hydroxyl, amino, halo, nitro, alkylamino, acyl, cyano, carboxy, aminocarbonyl, alkoxycarbonyl and aralkoxycarbonyl.

The term “heteroaryl”, alone or in combination means a five- or six-membered aromatic ring-containing moiety or a fused ring system (radical) containing two or three rings that have carbon atoms and also one or more heteroatoms in the ring(s) such as sulfur, oxygen and nitrogen. Examples of such heterocyclic or heteroaryl groups are pyrrolidinyl, piperidyl, piperazinyl, morpholinyl, thiamorpholinyl, pyrrolyl, imidazolyl (e.g., imidazol-4-yl, 1-benzyloxycarbonylimidazol-4-yl, and the like), pyrazolyl, pyridyl, pyrazinyl, pyrimidinyl, furyl, tetrahydrofuryl, thienyl, triazolyl, tetrazolyl, oxazolyl, oxadiazoyl, thiazolyl, thiadiazoyl, indolyl (e.g., 2-indolyl, and the like), quinolinyl, (e.g., 2-quinolinyl, 3-quinolinyl, 1-oxido-2-quinolinyl, and the like), isoquinolinyl (e.g., 1-isoquinolinyl, 3-isoquinolinyl, and the like), tetrahydroquinolinyl (e.g., 1,2,3,4-tetrahydro-2-quinolyl, and the like), 1,2,3,4-tetrahydroisoquinolinyl (e.g., 1,2,3,4-tetrahydro-1-oxo-isoquinolinyl, and the like), quinoxalinyl, β-carbolinyl, 2-benzofurancarbonyl, benzothiophenyl, 1-, 2-, 4- or 5-benzimidazolyl, and the like radicals.

The term “aralkyl”, alone or in combination, means an alkyl radical as defined above in which one hydrogen atom is replaced by an aryl radical as defined above, such as benzyl, diphenylmethyl, triphenylmethyl, phenylethyl, diphenylethyl 2-phenylethyl, and the like. The aryl in said aralkyl may be additionally substituted with halo, alkyl, alkoxy, halkoalkyl and haloalkoxy. The terms benzyl and phenylmethyl are interchangeable.

The term “aralkoxy”, alone or in combination, means an aralkyl radical attached through an oxygen atom to other radicals.

The term “aralkoxyalkyl”, alone or in combination, means an aralkoxy radical attached through an oxygen atom to an alkyl radical.

The term “aralkylthio”, alone or in combination, means an aralkyl radical attached to a sulfur atom.

The term “aralkylthioalkyl”, alone or in combination, means an aralkylthio radical attached through a sulfur atom to an alkyl radical.

The term “aralkoxycarbonyl”, alone or in combination, means a radical of the formula aralkyl-O—C(O)— in which the term “aralkyl” has the significance given above. An example of an aralkoxycarbonyl radical is benzyloxycarbonyl.

The term “aryloxy”, alone or in combination, means a radical of the formula aryl-O— in which the term aryl has the significance given above. The phenoxy radical is an exemplary aryloxy radical.

The term “aminoalkyl”, alone or in combination, means an alkyl radical substituted with amino radicals. Preferred are aminoalkyl radicals having alkyl portions having one to six carbon atoms. Examples of such radicals include aminomethyl, aminoethyl, and the like.

The term “alkylamino”, alone or in combination, means an amino group which has been substituted with one or two alkyl radicals. Preferred are N-alkylamino radicals having alkyl portions having one to six carbon atoms. Suitable alkylamino may be mono or dialkylamino such as N-methylamino, N-ethylamino, N,N-dimethylamino, N,N-diethylamino, and the like.

The term “arylamino”, alone or in combination, means an amino group which has been substituted with one or two aryl radicals, such as N-phenylamino. The “arylamino” radicals may be further substituted on the aryl ring portion of the radical.

The term “aralkylamino”, alone or in combination, means an aralkyl radical attached through a nitrogen atom to other radicals. The terms “N-arylaminoalkyl” and “N-aryl-N-alkyl-aminoalkyl” mean an amino group which have been substituted with one aryl radical or one aryl and one alkyl radical, respectively, and having the amino group attached to an alkyl radical. Examples of such radicals include N-phenylaminomethyl, N-phenyl-N-methylaminomethyl, and the like.

The terms “heteroaralkyl” and “heteroaryloxy”, alone or in combination, mean a radical structurally similar to aralkyl and aryloxy that are formed from heteroaryl radicals. Exemplary radicals include 4-picolinyl and 2-pyrimidinoxy, respectively.

The terms “alkanoyl” or “alkylcarbonyl”, alone or in combination, mean an acyl radical derived from an alkanecarboxylic acid, examples of which include formyl, acetyl, propionyl, butyryl, valeryl, 4-methylvaleryl, and the like.

The term “cycloalkylcarbonyl”, alone or in combination, means an acyl group derived from a monocyclic or bridged cycloalkanecarboxylic acid such as cyclopropanecarbonyl, cyclohexanecarbonyl, adamantanecarbonyl, and the like, or from a benz-fused monocyclic cycloalkanecarboxylic acid that is optionally substituted by, for example, alkanoylamino, such as 1,2,3,4-tetrahydro-2-naphthoyl, 2-acetamido-1,2,3,4-tetrahydro-2-naphthoyl.

The terms “aralkanoyl” or “aralkylcarbonyl”, alone or in combination, mean an acyl radical derived from an aryl-substituted alkanecarboxylic acid such as phenylacetyl, 3-phenylpropionyl (hydrocinnamoyl), 4-phenylbutyryl, (2-naphthyl)acetyl, 4-chlorohydrocinnamoyl, 4-aminohydrocinnamoyl, 4-methoxyhydrocinnamoyl, and the like.

The terms “aroyl” or “arylcarbonyl”, alone or in combination, mean an acyl radical derived from an aromatic carboxylic acid. Examples of such radicals include aromatic carboxylic acids, an optionally substituted benzoic or naphthoic acid such as benzoyl, 4-chlorobenzoyl, 4-carboxybenzoyl, 4-(benzyloxycarbonyl)benzoyl, 1-naphthoyl, 2-naphthoyl, 6-carboxy-2 naphthoyl, 6-(benzyloxycarbonyl)-2-naphthoyl, 3-benzyloxy-2-naphthoyl, 3-hydroxy-2-naphthoyl, 3-(benzyloxyformamido)-2-naphthoyl, and the like.

The terms “carboxy” or “carboxyl”, whether used alone or in combination, i.e., with other terms, such as “carboxyalkyl”, mean a —CO₂H radical.

The term “carboxyalkyl”, alone or in combination, means an alkyl radical substituted with a carboxy radical. More preferred carboxyalkyl radicals have alkyl radicals as defined above, and may be additionally substituted on the alkyl radical with halo. Examples of such carboxyalkyl radicals include carboxymethyl, carboxyethyl, carboxypropyl, and the like.

The term “alkoxycarbonyl”, alone or in combination, means a radical containing an alkoxy radical, as defined above, attached via an oxygen atom to a carbonyl radical. More preferred alkoxycarbonyl radicals have alkyl portions having one to six carbons. Examples of such alkoxycarbonyl (ester) radicals include substituted or unsubstituted methoxycarbonyl, ethoxycarbonyl, propoxycarbonyl, butoxycarbonyl, hexyloxycarbonyl, and the like.

The term “cycloalkylalkoxycarbonyl”, alone or in combination, means an acyl group of the formula cycloalkylalkyl-O—CO— wherein cycloalkylalkyl has the significance given above.

The term “aryloxyalkanoyl”, alone or in combination, means an acyl radical of the formula aryl-O-alkanoyl wherein aryl and alkanoyl have the significance given above.

The term “heterocyclooxycarbonyl”, alone or in combination, means an acyl group having the formula heterocyclo-O—CO— wherein heterocyclo is as defined above.

The term “heterocycloalkanoyl”, alone or in combination, means an acyl radical of the formula heterocyclo-substituted alkane carboxylic acid wherein heterocyclo has the significance given above.

The term “heterocycloalkoxycarbonyl”, alone or in combination, means an acyl radical of the formula heterocyclo-substituted alkane-O—CO— wherein heterocyclo has the significance given above.

The term “heteroaryloxycarbonyl”, alone or in combination, means an acyl radical represented by the formula heteroaryl-O—CO— wherein heteroaryl has the significance given above.

The term “aminocarbonyl” (carboxamide) alone or in combination, means an amino-substituted carbonyl (carbamoyl) group derived from an amine reacted with a carboxylic acid wherein the amino (amido nitrogen) group is unsubstituted (—NH₂) or a substituted primary or secondary amino group containing one or more substituents selected from hydrogen, alkyl, aryl, aralkyl, cycloalkyl, cycloalkylalkyl, and the like, as recited. A hydroxamate is a N-hydroxycarboxamide.

The term “alkylaminoalkyl”, alone or in combination, means a radical having one or more alkyl radicals attached to an aminoalkyl radical.

The term “aryloxyalkyl”, alone or in combination, means a radical having an aryl radical attached to an alkyl radical through a divalent oxygen atom.

The term “arylthioalkyl”, alone or in combination, means a radical having an aryl radical attached to an alkyl radical through a divalent sulfur atom.

The term “aminoalkanoyl”, alone or in combination, means an acyl group derived from an amino-substituted alkanecarboxylic acid wherein the amino group can be a primary or secondary amino group containing substituents independently selected from hydrogen, alkyl, aryl, aralkyl, cycloalkyl, cycloalkylalkyl, and the like.

The term “aromatic ring” in combinations such as substituted-aromatic ring sulfone or substituted-aromatic ring sulfoxide means aryl or heteroaryl as defined before.

The term “pharmaceutically acceptable” is used adjectivally herein to mean that the modified noun is appropriate for use in a pharmaceutical product. Pharmaceutically acceptable cations include metallic ions and organic ions. More preferred metallic ions include, but are not limited to appropriate alkali metal (Group Ia) salts, alkaline earth metal (Group IIa) salts and other physiological acceptable metal ions. Exemplary ions include aluminum, calcium, lithium, magnesium, potassium, sodium and zinc in their usual valences. Preferred organic ions include protonated tertiary amines and quaternary ammonium cations, including in part, trimethylamine, diethylamine, N,N′-dibenzylethylenediamine, chloroprocaine, choline, diethanolamine, ethylenediamine, meglumine (N-methylglucamine) and procaine. Exemplary pharmaceutically acceptable acids include without limitation hydrochloric acid, hydrobromic acid, phosphoric acid, sulfuric acid, methanesulfonic acid, acetic acid, formic acid, tartaric acid, maleic acid, malic acid, citric acid, isocitric acid, succinic acid, lactic acid, gluconic acid, glucuronic acid, pyruvic acid oxalacetic acid, fumaric acid, propionic acid, aspartic acid, glutamic acid, benzoic acid, and the like.

Nonlimiting examples of cyclooxygenase-2 inhibitors that may be used in the methods, combinations and compositions of the present invention are identified in Table 1 below.

TABLE 1
Some Individual Cyclooxygenase-2 Inhibitors Useful in the Present
Invention.

| | Trade | | |
|---|---|---|---|
| Compound | Name | Reference | Dosage |
| 6-chloro-4-hydroxy-2- | lornoxicam; | CAS No. | |
| methyl-N-2-pyridinyl-2H- | Safem ® | 70374-39-9 | |
| thieno[2,3-e]-1,2-thiazine-3- | | | |
| carboxamide, 1,1-dioxide | | | |
| 1,5-Diphenyl-3-substituted | | WO 97/13755 | |
| pyrazoles | | | |
| | radicicol | WO 96/25928; | |
| | | Kwon et al | |
| | | (Cancer | |
| | | Res(1992) 52 | |
| | | 6296) | |
| | GB- | | |
| | 02283745 | | |
| | TP-72 | Cancer Res. | |
| | | 1998 58 4 717 | |
| | | -723 | |
| 1-(4-chlorobenzoyl)-3-[4-(4- | A-183827.0 | | |
| fluorophenyl )thiazol-2- | | | |
| ylmethyl]-5-methoxy-2- | | | |
| methy lindole | | | |
| | GR-253035 | CAS Registry | |
| | | No. 215522- | |
| | | 99-9 | |
| 4-(4-cyclohexyl-2- | JTE-522 | CAS Registry | |
| methyloxazol-5-yl)-2- | | Number: | |
| fluorobenzenesulfonamide; | | 180200-68-4; | |
| Benzenesulfonamide, 4-(4- | | JP 09052882 | |
| cyclohexyl-2-methyl-5- | | | |
| oxazolyl)-2-fluoro- | | | |
| 5-chloro-3-(4- | | | |
| (methylsulfonyl)phenyl)-2- | | | |
| (methyl-5-pyridinyl)pyridine | | | |
| 2-(3,5-difluorophenyl)-3-4- | | | |
| (methylsulfonyl)phenyl)-2- | | | |
| cyclopenten-1-one | | | |
| 5-[4-(methylsulfonyl)- | L-768277 | CAS Registry | |
| phenyl]-6-phenyl- | | No. 180696- | |
| thiazolo[3,2-b][1,2,4]triazole | | 49-5 | |
| | L-783003 | CAS Registry | |
| | | No. 215435- | |
| | | 69-1 | |
| 4-(4-(methyl- | MK-966; | U.S. Pat. No. 5,968,974 | 12.5-100 mg po |
| sulfonyl)phenyl]-3-phenyl- | Vioxx ®; | | |
| 2(5H)-furanone; | rofecoxib | | |
| indomethacin-derived | | WO 96/37467- | 200 mg/kg/day |
| indolalkanoic acid | | 9 | |
| 1-Methylsulfonyl-4-[1,1- | | WO 95/30656; | |
| dimethyl-4-(4- | | WO 95/30652; | |
| fluorophenyl)cyclopenta-2,4- | | WO 96/38418; | |
| dien-3-yl]benzene | | WO 96/38442 | |
| 4,4-dimethyl-2-phenyl-3-[4- | | | |
| (methylsulfonyl)phenyl]cycl | | | |
| obutenone | | | |
| 2-(4-methoxyphenyl)-4- | | EP 799823 | |
| methyl-1-(4- | | | |
| sulfamoylphenyl)pyrrole | | | |
| N-[5-(4- | RWJ-63556 | | |
| fluoro)phenoxy]thiophene-2- | | | |
| methanesulfonamide | | | |
| 5(E)-(3,5-di-tert-butyl-4- | S-2474 | EP 595546 | |
| hydroxy)benzylidene-2- | | | |
| ethyl-1,2-isothiazolidine-1,1- | | | |
| dioxide | | | |
| 3-formylamino-7- | T-614 | DE 3834204 | |
| methylsulfonylamino-6- | | | |
| phenoxy-4H-1-benzopyran- | | | |
| 4-one | | | |
| Benzenesulfonamide, 4-(5- | celecoxib; | CAS Registry | |
| (4-methylphenyl)-3- | Celebrex ® | Number: | |
| (trifluoromethyl)-1H- | | 169590-42-5; | |
| pyrazol-1-yl)- | | U.S. Pat. No. 5,466,823 | |
| Benzenesulfonamide, 4-(5- | valdecoxib | CAS Registry | |
| methyl-3-phenyl-4- | | Number: | |
| isoxazolyl)- | | 181695-72-7; | |
| | | 5,633,272 | |
| Propanamide, N-[[4-(5- | parecoxib | CAS Registry | |
| methyl-3-phenyl-4- | (prodrug) | Number: | |
| isoxazolyl)phenyl]sulfonyl]- | | 198470-84-7; | |
| | | U.S. Pat. No. 5,932,598 | |
| 4-[5-(3-fluoro-4- | deracoxib | CAS Registry | |
| methoxyphenyl)-3- | | Number: | |
| difluoromethyl)-1H-pyrazol- | | 169590-41-4; | |
| 1-yl]benzenesulfonamide | | U.S. Pat. No. 5,521,207 | |
| | meloxicam | U.S. Pat. No. 4,233,299 | 15-30 mg/day |
| | nimesulide | U.S. Pat. No. 3,840,597 | |
| 1,5-Diphenyl-3-substituted | | WO 97/13755 | |
| pyrazoles | | | |
| | radicicol | WO 96/25928. | |
| | | Kwon et al | |
| | | (Cancer | |
| | | Res(1992) 52 | |
| | | 6296) | |
| | TP-72 | Cancer Res. | |
| | | 1998 58 4 717 | |
| | | -723 | |
| 1-(4-chlorobenzoyl)-3-[4-(4- | A-183827.0 | | |
| fluoro-phenyl )thiazol-2- | | | |
| ylmethyl]-5-methoxy-2- | | | |
| methy lindole | | | |
| | GR-253035 | | |
| 5-chloro-3-(4- | | | |
| (methylsulfonyl)phenyl)-2- | | | |
| (methyl-5-pyridinyl)- | | | |
| pyridine | | | |
| 2-(3,5-difluoro-phenyl)-3-4- | | | |
| (methylsulfonyl)-phenyl)-2- | | | |
| cyclopenten-1-one | | | |
| CS 502 | Sankyo | | |
| 2-(6-methylpyrid-3-yl)-3-(4- | etoricoxib; | WO 98/03484; | |
| methylsulfinylphenyl)-5- | MK-663; L- | Bioorg. Med. | |
| chloropyridine | 791456 | Chem. Lett. | |
| | | 1998, 8, 2777- | |
| | | 2782 | |

The following individual references listed in Table No. 2 below, each hereby incorporated by reference, describe various cyclooxygenase-2 inhibitors suitable for use in the methods, combinations and compositions of the present invention described herein, and processes for their manufacture.

TABLE NO 2
Some Individual Cyclooxygenase-2 Inhibitor References

| | | | |
|---|---|---|---|
| WO 99/30721 | WO 99/30729 | U.S. Pat. No. 5,760,068 | WO 98/15528 |
| WO 99/25695 | WO 99/24404 | WO 99/23087 | FR 27/71005 |
| EP 921119 | FR 27/70131 | WO 99/18960 | WO 99/15505 |
| WO 99/15503 | WO 99/14205 | WO 99/14195 | WO 99/14194 |
| WO 99/13799 | GB 23/30833 | U.S. Pat. No. 5,859,036 | WO 99/12930 |
| WO 99/11605 | WO 99/10332 | WO 99/10331 | WO 99/09988 |
| U.S. Pat. No. 5,869,524 | WO 99/05104 | U.S. Pat. No. 5,859,257 | WO 98/47890 |
| WO 98/47871 | U.S. Pat. No. 5,830,911 | U.S. Pat. No. 5,824,699 | WO 98/45294 |
| WO 98/43966 | WO 98/41511 | WO 98/41864 | WO 98/41516 |
| WO 98/37235 | EP 86/3134 | JP 10/175861 | U.S. Pat. No. 5,776,967 |
| WO 98/29382 | WO 98/25896 | ZA 97/04806 | EP 84/6,689 |
| WO 98/21195 | GB 23/19772 | WO 98/11080 | WO 98/06715 |
| WO 98/06708 | WO 98/07425 | WO 98/04527 | WO 98/03484 |
| FR 27/51966 | WO 97/38986 | WO 97/46524 | WO 97/44027 |
| WO 97/34882 | U.S. Pat. No. 5,681,842 | WO 97/37984 | U.S. Pat. No. 5,686,460 |
| WO 97/36863 | WO 97/40012 | WO 97/36497 | WO 97/29776 |
| WO 97/29775 | WO 97/29774 | WO 97/28121 | WO 97/28120 |
| WO 97/27181 | WO 95/11883 | WO 97/14691 | WO 97/13755 |
| WO 97/13755 | CA 21/80624 | WO 97/11701 | WO 96/41645 |
| WO 96/41626 | WO 96/41625 | WO 96/38418 | WO 96/37467 |
| WO 96/37469 | WO 96/36623 | WO 96/36617 | WO 96/31509 |
| WO 96/25405 | WO 96/24584 | WO 96/23786 | WO 96/19469 |
| WO 96/16934 | WO 96/13483 | WO 96/03385 | U.S. Pat. No. 5,510,368 |
| WO 96/09304 | WO 96/06840 | WO 96/06840 | WO 96/03387 |
| WO 95/21817 | GB 22/83745 | WO 94/27980 | WO 94/26731 |
| WO 94/20480 | WO 94/13635 | FR 27/70,131 | U.S. Pat. No. 5,859,036 |
| WO 99/01131 | WO 99/01455 | WO 99/01452 | WO 99/01130 |
| WO 98/57966 | WO 98/53814 | WO 98/53818 | WO 98/53817 |
| WO 98/47890 | U.S. Pat. No. 5,830,911 | U.S. Pat. No. 5,776,967 | WO 98/22101 |
| DE 19/753463 | WO 98/21195 | WO 98/16227 | U.S. Pat. No. 5,733,909 |
| WO 98/05639 | WO 97/44028 | WO 97/44027 | WO 97/40012 |
| WO 97/38986 | U.S. Pat. No. 5,677,318 | WO 97/34882 | WO 97/16435 |
| WO 97/03678 | WO 97/03667 | WO 96/36623 | WO 96/31509 |
| WO 96/25928 | WO 96/06840 | WO 96/21667 | WO 96/19469 |
| U.S. Pat. No. 5,510,368 | WO 96/09304 | GB 22/83745 | WO 96/03392 |
| WO 94/25431 | WO 94/20480 | WO 94/13635 | JP 09052882 |
| GB 22/94879 | WO 95/15316 | WO 95/15315 | WO 96/03388 |
| WO 96/24585 | U.S. Pat. No. 5,344,991 | WO 95/00501 | U.S. Pat. No. 5,968,974 |
| U.S. Pat. No. 5.945.539 | U.S. Pat. No. 5,994,381 | U.S. Pat. No. 5,521,207 | U.S. Pat. No. 6,310,099 |
| U.S. Pat. No. 6,291,523 | U.S. Pat. No. 5,958,978 | WO 02/20090 | |

The rofecoxib used in the therapeutic methods, combinations and compositions of the present invention can be prepared in the manner set forth in U.S. Pat. No. 5,968,974.

Celecoxib can be prepared in the manner set forth in U.S. Pat. No. 5,466,823.

Valdecoxib can be prepared in the manner set forth in U.S. Pat. No. 5,633,272.

Parecoxib can be prepared in the manner set forth in U.S. Pat. No. 5,932,598.

Deracoxib can be prepared in the manner set forth in U.S. Pat. No. 5,521,207.

JTE-522 can be prepared in the manner set forth in JP 90/52,882.

Etoricoxib can be prepared in the manner set forth in PCT Patent Application No. WO 98/03484.

Lumiracoxib can be prepared in the manner set forth in WO 02/20090.

The following examples illustrate embodiments of the invention but are not to be taken as a limiting of the scope of the invention as described and claimed herein.

### EXAMPLES

To determine the consequences of selective COX inhibition on weight loss and hypophagia after induction of systemic inflammation with LPS, adult mice were treated with LPS and selective or non-selective COX inhibitors.

Animal housing. Wild type (WT), COX-1-deficient (KO) mice (Langenbach et al., Cell 83: 483-492, 1995), and COX-2 KO mice (Morham et al., Cell 83: 473-482; 1995) were housed on a 12 h:12 h light:dark cycle with ad libitum access to rodent chow unless otherwise indicated. For pharmacological inhibitor studies, male C3H/HeN mice (Harlan Sprague Dawley, Inc., Indianapolis, Ind.) 10-12 wks of age were evaluated according to the method of Gross et al., Am J Physiol 278: R1415-23, 2000. See also Silver et al., J Clin Invest 95: 725-31, 1995. Male COX-1 and COX-2 KO mice, evaluated at 8-14 weeks of age, were of an outbred C57BL/6×129 background maintained by KO male×heterozygote female matings for the COX-1 KO allele and heterozygote×heterozygote matings for the COX-2 KO allele. Control mice designated “WT” for the COX KO experiments were WT and heterozygous littermates arising from the COX-2 heterozygous matings. All mouse protocols were in accordance with NIH guidelines and approved by the Animal Care and Use Committee of Washington University School of Medicine.

LPS-induced anorexia. C3H/HeN male mice were singly housed and acclimatized for at least 2 days to cages with grid bottoms prior to treatments. Body weight and food intake was measured daily until a stable baseline was obtained. Mice received either vehicle (phosphate buffered saline; PBS) or 100 μg of lipopolysaccharide (Escherichia coli 0111 :B4, Sigma, St. Louis, Mo.) in PBS via shallow midline, mid-abdominal, intraperitoneal injection. In our initial dose-response series, 100 μg LPS reliably induced weight loss following administration without mortality. To determine the effect of COX inhibition, mice were pretreated with either vehicle (phosphate buffered saline plus 1% Tween 80), or vehicle plus agent 30 minutes prior to i.p. injection of LPS or vehicle, and re-dosed at 8 and 24 hrs after LPS. Cyclooxygenase inhibitors were administered via gavage using olive-tipped needles. Food intake and weight loss were assessed in mice treated with the non-specific COX inhibitor indomethacin at 150 μg/dose (approximately 5 mg/kg), the COX-1-specific inhibitor Compound 2 (Pharmacia Corp., St. Louis, Mo.) at 300 μg/dose (10 mg/kg), and the COX-2 specific inhibitor Compound 1 (Pharmacia Corp., St. Louis, Mo.) at 300 μg/dose (10 mg/kg). Doses for the specific inhibitors were determined based on their reported IC₅₀ values for inhibition of COX-1 or COX-2 relative to indomethacin, and have previously been demonstrated to be isoform-selective. IC₅₀ values for inhibition of COX-1 or COX-2 relative to indomethacin and isoform selectivity have been reported in the following individual references.

a. Masferrer et al., Proc Natl Acad Sci USA 91: 3228-3232, 1994.

b. Penning et al., J Med Chem 40:1347-1365, 1997.

c. Smith et al., Proc Natl Acad Sci USA 95:13313-8, 1998.

d. Gross, et al., Am J Physiol 278: R1415-23, 2000.

Serum corticosterone, leptin, and IL-6 measurements. Plasma for measurement of corticosterone, leptin, and IL-6 was obtained from singly-housed male C3H/HeN mice (n=3-5 per group) by rapid retro-orbital phlebotomy into heparinized capillary tubes with a total time from first handling the animal to completion of bleeding not exceeding 30 s. The baseline, t=0, sample was obtained at 1000 h in ad libitum fed mice. At, 1400 h mice were administered vehicle or Compound 1 via gavage and food was removed from the cage for the remainder of the experiment. Mice underwent i.p. injection 30 min after vehicle or Compound 1 treatment with vehicle or LPS. Additional blood samples were then collected 3 and 16 h after the LPS injection in the fasted animals. Blood was collected on ice, and plasma separated by centrifugation and stored at −80° C. until assay. Plasma concentration of corticosterone (ICN Biomedicals, Inc., Costa Mesa, Calif.) was determined by radioimmunoassay as previously described for example by Bethin et al., Proc Natl Acad Sci USA 97: 9317-22, 2000. See also Jacobson et al., Endocrinology 138: 1048-57, 1997. Plasma concentrations of IL-6 (Pharmingen, San Diego, Calif.) and leptin (R & D Systems, Inc., Minneapolis, Minn.) were measured by ELISA per the manufacturer's instructions.

Hypothalamic in situ mRNA hybridization. Hypothalami were isolated from C3H/HeN male mice (n=3 per group) pre-treated with either vehicle or Compound 1 as described above at 1400 h, followed by injection of vehicle or LPS and food withdrawal for 16 h. After 24 h immersion in 4% paraformaldehyde, hypothalami were cryoprotected in 10% sucrose in DEPC D-PBS. Samples were embedded in OCT (Sakura Finetek USA, Inc., Torrance, Calif.), cut into 10 micron sections on a cryostat, and thaw mounted onto Superfrost plus slides (Fisher Scientific, Pittsburgh, Pa.). In situ hybridization utilized α-³³P-UTP-labelled 320 base anti-sense riboprobe from the mouse CRH gene or a 511 base anti-sense riboprobe from the rat neuropeptide Y (NPY) cDNA (generously provided by Dr. Steven Sabol, NHLBI, NIH) by methods described (e.g., Muglia et al., J Neurosci 19: 2051-8, 1999). For CRH and NPY mRNA detection, the regions of the hypothalamic paraventicular and arcuate nuclei were determined by Nissl staining and light microscopy. Six sections spanning each nucleus were hybridized to CRH (paraventricular nucleus) or NPY (arcuate nucleus) probes, and the two sections of peak intensity imaged for densitometric analysis. Hybridizing probes were quantitated by exposure of slides to Hyperfilm-βMax (Amersham Life Science, Inc., Arlington Heights, Ill.) with densitometric analysis employing NIH Image Software.

Statistical Methods. All results are expressed as mean ±SEM. Statistical analysis was by ANOVA, with p≦0.05 considered significant.

Results

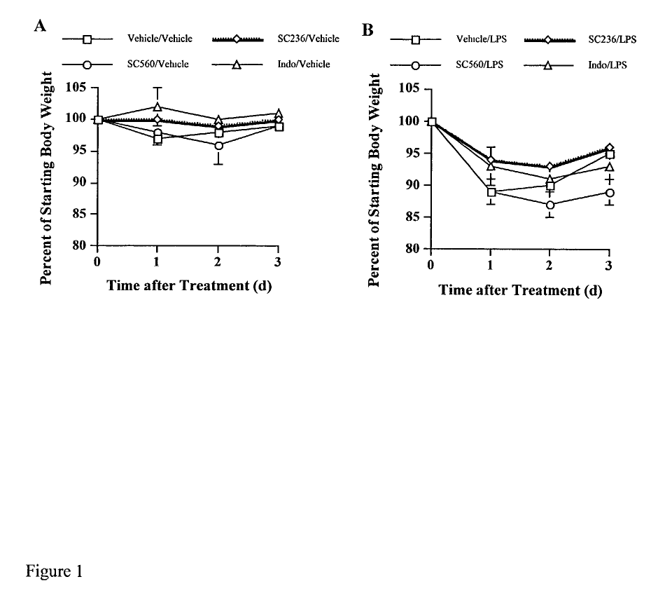

Administration of the COX-2-selective inhibitor Compound 1 (also known as SC236), the COX-1 -selective inhibitor Compound 2 (also known as SC560), or the non-selective inhibitor indomethacin without administration of LPS resulted in no change in body weight relative to vehicle-treated control mice (FIG. 1A).

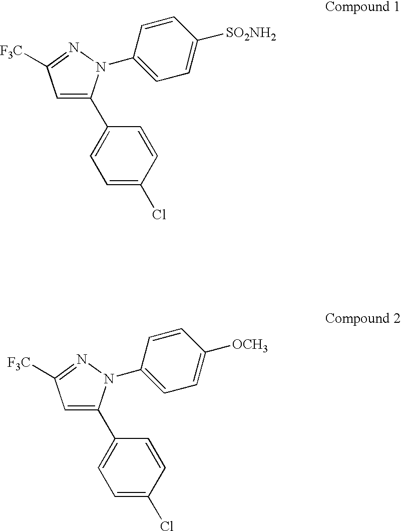

Treatment with indomethacin tended to reduce the weight loss associated with LPS administration (FIG. 1B, P=0.09 vs. vehicle/LPS at 1 day after treatment). Selective attenuation of COX-2 activity with Compound 1 significantly attenuated weight loss in comparison to both mice treated with vehicle and LPS or the Compound 2 and LPS (FIG. 1B). One day after LPS treatment, weight loss of mice receiving Compound 1 was approximately 50% of the vehicle/LPS and Compound 2/LPS groups. The difference in weight loss between LPS-treated Compound 1 and Compound 2 groups remained significant at 2 and 3 days after LPS treatment. Additionally, the Compound 2/LPS group exhibited sustained weight reduction in comparison to the vehicle/LPS group at 3 days after treatment, suggesting that inhibition of COX-1 during systemic inflammation may be detrimental.

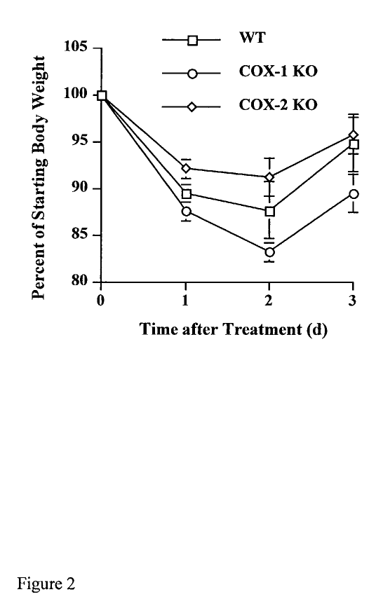

To further confirm the differential effects of COX-1- versus COX-2-derived prostaglandins on weight loss after systemic immune activation, WT, COX-1, and COX-2 KO mice following LPS administration were evaluated. For these experiments, female mice were used since COX-2 KO male mice have early mortality (21) precluding acquisition of adequate numbers of otherwise healthy adult COX-2 KO males for our studies. In accord with the above experiments utilizing COX-selective inhibitors, COX-2 KO mice demonstrated significantly less weight loss than COX-1 KO mice 1 day after LPS administration (P<0.01; FIG. 2), and tended to have less weight loss than WT mice at this time point as well (P=0.08). At two days after LPS administration, COX-2 KO mice continued to exhibit significantly better preservation of weight than COX-1 KO mice.

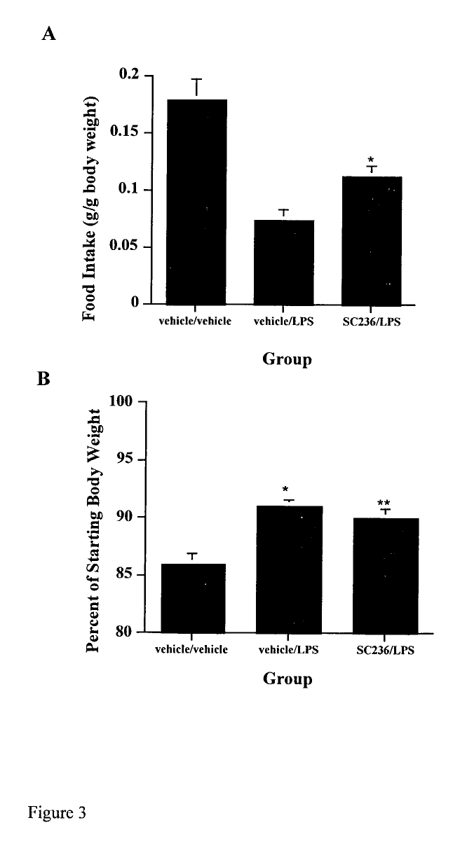

Changes in either energy intake or energy utilization could contribute to the attenuation in weight loss associated with selective COX-2 inhibition or deficiency. To evaluate the effect of COX-2 inhibition on appetite, food intake in LPS-treated mice with or without simultaneous COX-2 inhibition by Compound 1 was measured (FIG. 3A). Administration of LPS alone resulted in a 60% reduction in food intake in comparison to vehicle-treated mice over the 24 h period following induction of systemic immune system activation. In contrast, Compound 1 administration to LPS-treated animals attenuated the hypophagic response with food intake increasing by 50% in comparison to mice receiving LPS alone. Consistent with the attenuation of weight loss after LPS, Compound 1 attenuated, but did not completely block the hypophagic response. Further supporting that the effect of Compound 1 in reducing weight loss depends on maintenance of food intake rather than changes in energy utilization, mice that were treated with vehicle or Compound 1 and then fasted for 16 h following LPS administration demonstrated identical weight loss (FIG. 3B).

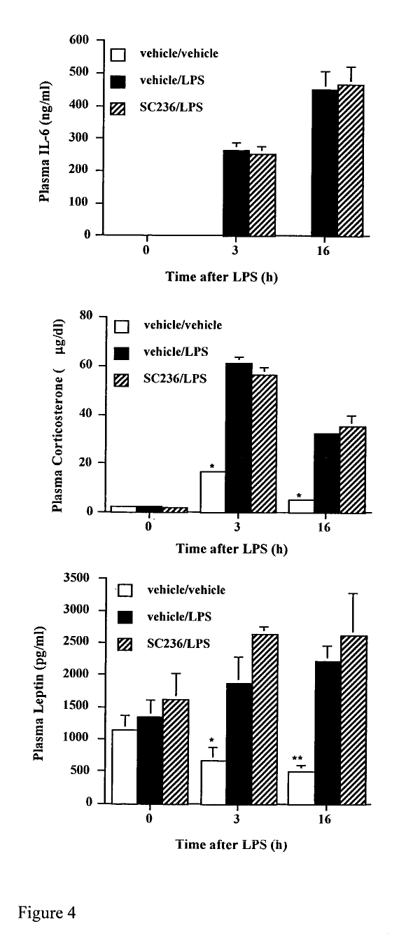

Maintenance of body weight, food intake, and overall nutritional status in the setting of acute bacterial infection or chronic inflammatory diseases would be of substantial health benefit. However, if maintenance of food intake and body weight after LPS occurred as a consequence of inhibition of the cytokine response to inflammation, the ability of tile organism to combat infection and not succumb to sepsis may be compromised. To determine the consequences of COX-2 inhibition on cytokine production, plasma IL-6 concentration was measured after LPS administration. Normal IL-6 production in response to LPS requires the prior induction of both TNF-α and IL-1β, and IL-6 remains elevated for a prolonged period, providing a useful marker for integration of the cytokine response (see for example Akira et al., FASEB J 4: 2860-7, 1990). As expected, IL-6 rose dramatically following LPS administration (FIG. 4A). Administration of Compound 1 in conjunction with LPS resulted in no change in the magnitude of early induction of IL-6 as measured 3 h after LPS, or sustained increases in IL-6 at 16 hours after LPS.

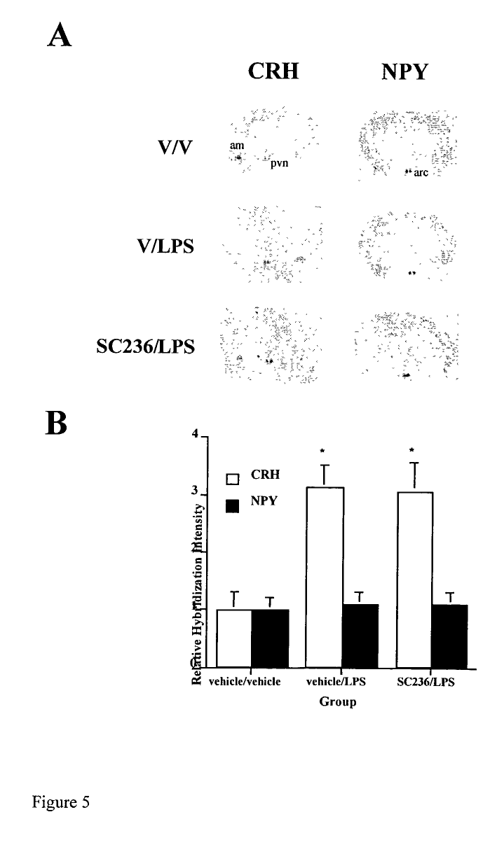

As additional evidence for the relative specificity of the consequences of COX-2 inhibition after LPS being on the hypophagic response rather than the overall cytokine and inflammatory response, the activity of the hypothalamic-pituitary-adrenal axis was measured. Stimulation of adrenal glucocorticoid release during inflammation reflects actions of cytokines at central nervous system, pituitary, and adrenal sites and provides a useful integrated measure of stimulation and damping of the inflammatory response. LPS administration resulted in marked elevation of plasma corticosterone concentrations at 3 h which was sustained at 16 h following administration (FIG. 4B). There was no difference in the magnitude or duration of adrenal activation in those mice treated with LPS and Compound 1. In accord with the sustained increase in adrenal glucocorticoid output after LPS, CRH mRNA in the paraventricular nucleus of the hypothalamus was elevated approximately 3-fold in both LPS and Compound 1/LPS groups (FIG. 5).

To determine whether COX-2-generated prostaglandins had direct effects on appetite or acted indirectly on appetite centers via modulation of neuropeptides or other molecules, measurements were made of neuropeptide Y mRNA in the arcuate nucleus of the hypothalamus and serum leptin concentration. Despite elevated cytokine levels and induction of the anorexic neuropeptide CRH, NPY levels did not differ in mice that received or did not receive LPS (FIG. 5). In contrast, serum leptin concentration was augmented by administration of LPS (FIG. 4C). The magnitude of induction of leptin did not differ between vehicle/LPS and Compound 1/LPS groups.

The data now presented demonstrate that selective pharmacological or genetic blockade of COX-2-generated prostaglandins effectively attenuates the hypophagic response to systemic inflammation induced by LPS. Unlike other agents that attenuate inflammation-induced anorexia, COX-2-selective inhibitors decrease anorexia without diminishing the cytokine response. The present results show no intermediate-to-long term reduction in IL-6 production. Without being limited to a specific mechanism, it is believed that because COX-2 inhibition does not alter the expression of anorexic neuropeptides such as CRH, orexic neuropeptides such as NPY, or leptin after LPS administration, COX-2-generated prostaglandins are likely to directly modulate central appetite centers. Alternatively, COX-2 may indirectly modulate appetite by action on one of several neuropeptide pathways.

In contrast to selective inhibition of COX-2, selective inhibition of COX-1 during inflammation appears detrimental. The present invention teaches that inhibition or genetic inactivation of COX-1 will prolong the time until recovery of baseline body weight after LPS injection, and combined inhibition of COX-1 and COX-2 will not be as effective as selective COX-2 inhibition in attenuating weight loss. Since transient COX-1 inhibition in control mice that did not receive LPS is not associated with weight loss, the present findings suggest that COX-1-generated prostaglandins serve a protective role during inflammation, such as maintaining gastric mucosal integrity in the face of fasting and stress. The majority of NSAIDs in common clinical use for fever control during infection demonstrate substantial COX-1 inhibitory activity, and are likely to cause similar decreases in food intake and body weight maintenance during the recovery phase of illness. The ability to selectively diminish COX-2-derived prostaglandins during inflammation or sepsis will maximize the beneficial actions of inhibiting inflammation-induced prostaglandins for fever control and food intake, maintain cytokine action, and minimize detrimental actions important for recovery from illness imparted by COX-1.

What is claimed is:

1. A method for the treatment or prophylaxis of a cachexic condition in a subject in need of such treatment or prevention, wherein the method comprises administering to the subject a cachexic condition treating- or preventing-effective amount of a cyclooxygenase-2 selective inhibiting compound, thereby treating or preventing the cachexic condition.

2. The method of claim 1 wherein the cyclooxygenase-2 selective inhibiting compound is a tricyclic cyclooxygenase-2 selective inhibiting compound.

3. The method of claim 1 wherein the tricyclic cyclooxygenase-2 selective inhibiting compound is selected from the group consisting of celecoxib, deracoxib, valdecoxib, parecoxib, rofecoxib, etoricoxib, and 4-(4-cyclohexyl-2-methyloxazol-5-yl)-2-fluorobenzenesulfonamide.

4. The method of claim 3 wherein the tricyclic cyclooxygenase-2 selective inhibiting compound is celecoxib.

5. The method of claim 3 wherein the tricyclic cyclooxygenase-2 selective inhibiting compound is deracoxib.

6. The method of claim 3 wherein the tricyclic cyclooxygenase-2 selective inhibiting compound is valdecoxib.

7. The method of claim 3 wherein the tricyclic cyclooxygenase-2 selective inhibiting compound is parecoxib.

8. The method of claim 3 wherein the tricyclic cyclooxygenase-2 selective inhibiting compound is rofecoxib.

9. The method of claim 3 wherein the tricyclic cyclooxygenase-2 selective inhibiting compound is etoricoxib.

10. The method of claim 1 wherein the cyclooxygenase-2 selective inhibiting compound is lumiracoxib.

11. The method of claim 3 wherein the tricyclic cyclooxygenase-2 selective inhibiting compound is 4-(4-cyclohexyl-2-methyloxazol-5-yl)-2-fluorobenzenesulfonamide.

12. The method of claim 1 wherein the cyclooxygenase-2 selective inhibiting compound is a benzopyran cyclooxygenase-2 inhibiting compound.

13. The method of claim 1 wherein the cyclooxygenase-2 selective inhibiting compound is selected from the group consisting of:
5-ethyl-2-(2′,4′-dichloro-6′-methylanilino)phenylacetic acid;
5-ethyl-2-(2′,3′,5′,6′-tetrafluoroanilino)phenylacetic acid;
5-methyl-2-(2′,4′-difluoro-6′-chloroanilino)phenylacetic acid; and
5-methyl-2-(2′-fluoro-6′-chloroanilino)phenylacetic acid.

14. The method of claim 1 wherein the cyclooxygenase-2 selective inhibiting compound is selected from the group consisting of:
celecoxib;
deracoxib;
valdecoxib;
parecoxib;
rofecoxib;
etoricoxib;
meloxicam;
lumiracoxib;
4-(4-cyclohexyl-2-methyloxazol-5-yl)-2-fluorobenzenesulfonamide;
6-[[5-(4-chlorobenzoyl)-1,4-dimethyl-1H-pyrrol-2-yl]methyl]-3(2H)-pyridazinone;
N-(4-nitro-2-phenoxyphenyl)methanesulfonamide;
3-(3,4-difluorophenoxy)-5,5-dimethyl-4-[4-(methylsulfonyl)phenyl]-2(5H)-furanone;
N-[6-[(2,4-difluorophenyl)thio]-2,3-dihydro-1-oxo-1H-inden-5-yl]methanesulfonamide;
N-[2-(cyclohexyloxy)-4-nitrophenyl]methanesulfonamide;
N-[6-(2,4-difluorophenoxy)-2,3-dihydro-1-oxo-1H-inden-5-yl]methanesulfonamide;
3-(4-chlorophenoxy)-4-[(methylsulfonyl)amino]benzenesulfonamide;
3-(4-fluorophenoxy)-4-[(methylsulfonyl)amino]benzenesulfonamide;
3 -[(1-methyl-1H-imidazol-2-yl)thio]-4[(methylsulfonyl) amino]benzenesulfonamide;
5,5-dimethyl-4-[4-(methylsulfonyl)phenyl]-3-phenoxy-2(5H)-furanone;
N-[6-[(4-ethyl-2-thiazolyl)thio]-1,3-dihydro-1-oxo-5-isobenzofuranyl]methanesulfonamide;
3-[(2,4-dichlorophenyl)thio]-4-[(methylsulfonyl)amino]benzene-sulfonamide;
N-(2,3 -dihydro-1,1-dioxido-6-phenoxy-1,2-benzisothiazol-5-yl)methanesulfonamide;
N-[3-(formylamino)-4-oxo-6-phenoxy-4H-1-benzopyran-7-yl]methanesulfonamide;
5-ethyl-2-(2′,4′-dichloro-6′-methylanilino)phenylacetic acid;
5-ethyl-2-(2′,3′,5′,6′-tetrafluoroanilino)phenylacetic acid;
5-methyl-2-(2′,4′-difluoro-6′-chloroanilino)phenylacetic acid; and
5-methyl-2-(2′-fluoro-6′-chloroanilino)phenylacetic acid.

15. The method of claim 1 wherein the cachexic condition is selected from the group consisting of cancer-related cachexia, hypophysiopriva-related cachexia, infectious disease-related cachexia, a cardiovascular-related cachexia, and anorexia.

16. The method of claim 1 wherein the cyclooxygenase-2 selective inhibiting compound has the following general formula: 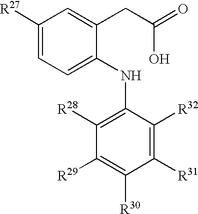
or an isomer, a pharmaceutically acceptable salt, ester, or prodrug thereof; wherein:
R²⁷ is methyl, ethyl, or propyl;
R²⁸ is chloro or fluoro;
R²⁹ is hydrogen, fluoro, or methyl;
R³⁰ is hydrogen, fluoro, chloro, methyl, ethyl, methoxy, ethoxy or hydroxy;
R³¹ is hydrogen, fluoro, or methyl; and
R³² is chloro, fluoro, trifluoromethyl, methyl, or ethyl, provided that R²⁸, R²⁹, R³⁰ and R³¹ are not all fluoro when R²⁷ is ethyl and R³⁰ is H.

17. The method of claim 16 wherein:
R²⁷ is methyl;
R²⁸ is fluoro;
R³² is chloro; and
R²⁹, R³⁰, and R³¹ are hydrogen.
